(19) United States
(12) Patent Application Publication
(10) Pub. No.: US 2011/0142817 A1
(43) Pub. Date: Jun. 16, 2011

(54) MEANS AND METHOD FOR TREATING AND/OR PREVENTING NECROTIZING ENTEROCOLITIS

(75) Inventors: Rudi BRANDS, Bunnik (NL); Klaas Poelstra, Buitenpost (NL)

(73) Assignee: PHARMAAWARE SEPSIS B.V., Bunnik (NL)

(21) Appl. No.: 12/830,812

(22) Filed: Jul. 6, 2010

Related U.S. Application Data
(63) Continuation-in-part of application No. 10/588,285, filed on Sep. 22, 2008.
(60) Provisional application No. 60/541,363, filed on Feb. 4, 2004.

(30) Foreign Application Priority Data
Feb. 4, 2004 (EP) 04075344.4

(51) Int. Cl. A61K 38/46; C12N 9/16; A61P 1/00

(52) U.S. Cl. 424/94.6; 435/196

(57) ABSTRACT

Compositions comprising a source of alkaline phosphatase that prevent or reduce toxic influx of lipopolysaccharide (LPS) through mucosal layers of a mamalian body cavity and methods of using these compositions for those purposes are disclosed. Such a source of alkaline phosphatase, preferably in a medical food such as infant milk formula, is eaten, drunk or otherwise administered for prophylaxis or treatment of LPS-mediated or LPS-exacerbated disease.

### BACKGROUND OF THE INVENTION

1. Field of the Invention

The current invention relates to the field of medicine and in particular to the use of LPS detoxifying and neutralizing enzymes. The present invention also relates to the field of pharmacy and in particular to the pharmaceutical use of alkaline phosphatase enzymes.

2. Description of the Background Art

Necrotizing enterocolitis (NEC) is a leading cause of morbidity and mortality in neonates, occurring in 0.3 to 2.4 per 1000 live births. Multiple risk factors for developing NEC have been identified, including prematurity, ischemia, formula feeding, and bacterial colonization of the intestine. Despite known risk factors and improved neonatal care, the management of NEC remains supportive and the mortality rate for the past 3 decades remains as high as 50%. Prevention, therefore, is a promising strategy to help reduce morbidity and mortality in NEC.

Although the pathogenesis of NEC remains incompletely understood, current thinking suggests that this disease develops in the stressed, premature host, after a disruption in the intestinal barrier, which leads to the translocation of Lipopolysaccharides (LPS). The potential importance of LPS in the pathogenesis of NEC is highlighted by studies that show that circulating levels of LPS are elevated in patients with NEC and that animal models of NEC are associated with increased levels of LPS in the plasma and stool. An increase in the circulating concentration of LPS may exert deleterious effects on the intestinal epithelial monolayer, characterized by a reduction in barrier integrity, while concomitantly activating the subepithelial leukocytes, leading to the proinflammatory cytokine release that characterizes NEC (Leaphart C L et al., J. Immunol. 2007).

LPS (also referred to as endotoxins) are present in the cell walls of Gram-negative bacteria. When LPS is presented to a vertebrate body it stimulates the innate and cellular immune responses in a wide variety of cell types. The production of cytokines and chemokines (such as TNF's, various interleukins, interferons and others) will attract and activate cells of the immune system, which may culminate ultimately in an LPS induced systemic inflammatory response syndrome (SIRS) under certain conditions.

LPS or endotoxins are toxic to most mammals and regardless of the bacterial source, all endotoxins produce the same range of biological effects in the animal host. The injection of living or killed Gram-negative cells, or purified LPS, into experimental animals causes a wide spectrum of non-specific pathophysiological reactions such as: fever, tachycardia, tachypneu, hyper or hypothermia, changes in white blood cell counts, disseminated intravascular coagulation, hypotension, organ dysfunction and may even result in shock and death.

Injection of small doses of endotoxin results in a proinflammatory response in most mammals, but the dose response range and steepness thereof varies significantly with the species and even within species may differ significantly depending on e.g., LPS-tolerance. The sequence of pro-inflammatory events follows a regular pattern (inflammatory cascade): (1) latent period; (2) physiological distress (diarrhea, prostration, shock); and in case of severe septic shock and multiple organ failure may result in (3) death. How soon death occurs varies on the dose of the endotoxin, route of administration, and species of animal.

The physiological effects of endotoxin are mainly mediated by the lipid A-moiety of LPS. Since Lipid A is embedded in the outer membrane of bacterial cells, it only exerts its toxic effects when released from multiplying cells in a soluble form, or when the bacteria are lysed as a result of autolysis, complement and the membrane attack complex (MAC), ingestion and killing by phagocytes, or killing with certain types of antibiotics. LPS released into the bloodstream can be neutralized by many blood components to a certain degree, amongst which several plasma lipids and proteins, among which LPS-binding proteins. The LPS-binding protein complex interacts with CD14 and Toll like receptors on monocytes and macrophages and through other receptors on endothelial cells. In monocytes and macrophages three types of events are triggered during their interaction with LPS:

Firstly, production of cytokines, including IL-1, IL-6, IL-8, tumor necrosis factor (TNF) and platelet-activating factor. These in turn stimulate production of prostaglandins and leukotrienes. These are powerful mediators of inflammation and septic shock that accompanies endotoxin toxemia. LPS activates macrophages to enhanced phagocytosis and cytotoxicity. Macrophages are stimulated to produce and release lysosomal enzymes, IL-1 (“endogenous pyrogen”), and tumor necrosis factor (TNF-α), as well as other cytokines and mediators.

Secondly, activation of the complement cascade. C3a and C5a cause histamine release (leading to vasodilation) and effect neutrophil chemotaxis and accumulation. The result is inflammation.

Finally, activation of the coagulation cascade. Initial activation of Hageman factor (blood-clotting Factor XII) can activate several humoral systems resulting in coagulation: a blood clotting cascade that leads to coagulation, thrombosis, acute disseminated intravascular coagulation, which depletes platelets and various clotting factors resulting in internal bleeding and also activation of the complement alternative pathway (as above, which leads to inflammation). Plasmin is activated which leads to fibrinolysis and hemorrhaging and kinin activation releases bradykinins and other vasoactive peptides which causes hypotension. The net effect is induction of inflammation, intravascular coagulation, hemorrhage and shock.

LPS also acts as a B cell mitogen stimulating the polyclonal differentiation and multiplication of B-cells and the secretion of immunoglobulins, especially IgG and IgM.

The physiological activities of LPS are mediated mainly by the Lipid A component of LPS. Lipid A is a powerful biological response modifier that can stimulate the mammalian immune system. During infectious disease caused by Gram-negative bacteria, endotoxins released from, or part of, multiplying cells have similar effects on animals and significantly contribute to the symptoms and pathology of the disease encountered. The primary structure of Lipid A has been elucidated and Lipid A has been chemically synthesized. Its biological activity appears to depend on a peculiar conformation that is determined by the glucosamine disaccharide, the PO₄ groups, the acyl chains, and also the KDO-containing inner core of the LPS molecule.

Alkaline phosphatase (AP), has been described earlier as a key enzyme in the dephosphorylation of LPS (endotoxin) under physiological conditions both in vitro and in vivo as a natural response to detoxify and neutralize LPS (U.S. Pat. No. 6,290,952, Poelstra et al., Am J. Pathol. 1997 October; 151(4):1163-9).

Reports on the enzyme activity of AP involve its extremely high pH optimum for the usual exogenous substrates and its localization as an ecto-enzyme. Endotoxins are molecules that contain several phosphate groups and are usually present in the extracellular space. AP is able to dephosphorylate this bacterial product at physiological pH levels, by removing phosphate groups from amongst others the toxic lipid A moiety of LPS. As phosphate residues in the lipid A moiety determine the toxicity of the molecule, the effect of the AP inhibitor levamisole in vivo using a septicemia model in the rat confirmed the specificity of AP for LPS containing phosphate groups (Poelstra et al., 1997). The results demonstrated that inhibition of endogenous AP by levamisole significantly reduces survival of rats intraperitoneally injected with E. coli bacteria, whereas this drug does not influence survival of rats receiving a sublethal dose of the gram-positive bacteria Staphylococcus aureus, illustrating a crucial role for this enzyme in host defense. The effects of levamisole during gram-negative bacterial infections and the localization of AP as an ecto-enzyme in most organs as well as the induction of enzyme activity during inflammatory reactions and cholestasis is in accordance with such a protective role.

The prime source of LPS exposure in the human body are the gram negative microorganisms that live within the human digestive or gastrointestinal (GI) tract. There are far more bacteria in the digestive system than there are on the skin or other parts of the body, making the GI tract and GI mucosa the main route of entry for LPS into the circulation. An average adult carries about 100 trillion bacteria in the intestines, most of which locate in the colon, contributing to 1-1.5 kg of his body weight. There are more than 400 species of bacteria found in the digestive system. These include both beneficial (commensal) and potentially harmful (pathogenic) species, which continually compete to maintain a well-balanced intestinal flora.

Mucosal surfaces, and in particular (but not limited to) the intestinal mucosa, are exposed to this wide variety of commensal and potentially pathogenic bacteria, among which many gram negative endotoxin/LPS producing, Gram-negative bacteria such as E. coli, Salmonella, Shigella, Pseudomonas, Neisseria, Haemophilus, Helicobacter, Chlamydia and other leading pathogens. The intestinal epithelium is of particular importance as it forms a dynamic barrier that regulates absorption of nutrients and water and at the same time restricts uptake of microbes and other noxious materials such as LPS from the gut lumen.

It is well established that a major fraction of LPS influx from the lumen of the gut through the mucosal lining into the circulation of a vertebrate body is mediated through chylomicrons (Harris et al., 1998, 2000, 2002). Coincidental with ingestion of lipids and chylomicron introduction in circulation, capable of carrying LPS, a significant increase in lymphatic AP derived from the GI-tract is reported (Nauli et al., 2002). LPS-influx through the GI-barrier is increased normally with a saturated fat-rich diet. LPS inserts with its lipid A acyl chain into lipoprotein phospholipids. Thereby LPS passes the intestinal barrier by co-migrating with chylomicrons, that are taken up predominantly at the small intestines ileum (Harris et al., 2002). After a fat rich food intake a significant rise of glycosyl-phosphatidyl-inositol (GPI)-anchored AP complexed to lipoproteins is detected in lymph as well (Nauli et al., 2003).

The physiological roles of—and the interpretation of AP serum levels are not clear, but a role in detoxification of LPS has emerged from current research. The co-presence of both AP and LPS in chylomicron rich fractions suggests a role for AP in dephosphorylating the gut derived-LPS already at close vicinity. Detoxification can take place both in the intestinal lumen or en-route to or upon presentation to the liver, specifically in this context to Kuppfer cells and the hepatocytes, which clear the chylomicrons from circulation.

Increased serum AP levels are associated with hepatic damage. Upon an endotoxin insult, circulatory AP is redirected to hepatocytes, thereby reducing circulating AP levels initially (Bentala et al., 2002) through receptor-mediated uptake (asialo-glycoprotein receptor). Hepatocytes also remove the LPS-loaded chylomicrons (Harris et al., 2002) rapidly from circulation with a half life of 5-10 minutes. LPS is next removed through biliary excretion, thereby preventing Kupffer cells, being a major target for circulating LPS to become activated (Harris, 2002). Bentala et al., 2002, showed that Kupffer cells accumulate AP in LPS-insulted animal models as well. This may imply that under normal conditions Kuppfer cells will not be activated since LPS (lipid A moiety), or its derivative MPLS (MPLA, derivative from Lipid A), is primarily presented to hepatocytes through a lipoprotein receptor and next is removed via biliary secretion. However under conditions with excess LPS, Kupffer cells are activated through a TLR-4 (LPS) receptor.

A wide array of animals have AP and several other entities present to counteract a (bacterial) insult, either local or systemic, induced or available as guard/watchdog function. Amongst others activated neutrophils or macrophages express a wide array of inflammatory mediators destined to neutralize the insult. Moieties like, but not restricted to LPS binding protein (LBP), CD14, Apo-E, VLDL, HDL, albumin, immunoglobulin and AP all have been described to serve this function. When such an insult however is not overcome, e.g., in case of a severe Gram negative or positive insult, resulting inflammatory mediators may initiate a systemic inflammatory response syndrome (SIRS).

It was postulated that AP is consumed as a consequence of its catalytic action towards LPS (Poelstra et al., 1997). This implies that subsequently normal levels are to be restored through a controlled mechanism. In patients suffering from septicemia, it has been observed that increased serum AP may be preceded by reduced AP serum levels (Manintveld and Poelstra, patent application EP 989626940) and that circulating AP would be cleared from circulation upon LPS interaction (Bentala et al., 2002). The increase in subsequent AP-levels therefore may be a feedback mechanism in response to this AP reduction. A mechanism for such a LPS/AP responsiveness has not been depicted to-date.

In inflammatory processes (temporary) increases are found for serum AP. In the context of this invention such an increase of AP is regarded as a natural response of the innate immune system to an LPS insult to tackle these insults and restore natural balance. Increased AP plasma levels are the result of massive shedding of AP from hepatocytes in response to the LPS insult. It has been observed that LPS induces Phospholipase-D activity (Locati et al., 2001) which in turn has been reported to act upon GPI anchored proteins, amongst which AP (Deng et al., 1996) and e.g., CD14, thereby effectively shedding the proteins into circulation (Zhang F et al., 2001, Locati M. et al., 2001).

Circulating plasma AP—predominantly anchorless liver type AP (Ahn et al., 2001)—may thus already have exerted its LPS detoxifying activity at the plasma membrane surface and is subsequently freed from the hepatocyte membrane into circulation prior to its subsequent elimination from circulation by e.g., the asialo glycoprotein-route.

AP exerts its catalytic activity towards LPS primarily in the vicinity of a membrane, possibly in so-called lipid rafts (“drm” or detergent-resistant membrane fraction) where it has been reported to reside. Several publications favor such a catalytic activity of AP at a membrane surface, either presented at the tissue level or released into circulation like with circulating liver plasma membrane fragments (LPMF) (e.g., Deng et al., 1996). The increased AP levels observed in chronically inflamed patients may be caused by the suboptimal detoxification of the gut-derived influx of LPS, which is often enhanced under pathological conditions prior to mobilization of hepatic AP.

The treatment of inflammatory diseases accounts for a substantial percentage of the gross medical cost in developed countries and the incidence of these inflammatory diseases is continuously rising due to key factors like ageing of the population and an increasing number of patients having suppressed immune systems as a consequence of medication and treatment of a wide array of diseases like heart disease, auto-immunity disorders and allergies, organ transplantations, cancer chemo- or radiotherapy and infectious diseases like AIDS. To a certain extent these diseases relate to an influx of bacterial LPS. The influx of LPS is often enhanced by a medical condition of a subject, causing an inflammatory process by a malfunctioning or non-balanced innate immune system, which constitutes the first line of defense against e.g., microbial insults, in particular from LPS/endotoxin producing bacteria.

For NEC, as said before, prevention is a promising strategy to help reduce morbidity and mortality. Many studies have focused on preventative measures, including the use of breast milk (Lucas A et al., Lancet 1990; Schanler R J et al., Pediatrics 1999) and growth factors (Dvorak B et al., Am J Physiol Gastrointest Liver Physiol 2002; Claud E C et al., Pediatr Res 2003). More recently, probiotics have been shown to decrease the incidence of NEC in very low birth weight infants in 2 randomized controlled trials (Bin-Nun A et al., J Pediatr 2005; Lin H C et al., Pediatrics 2008). Although the results of these studies are compelling, the optimal dosing regimen, duration of therapy, and bacterial strain to be used in probiotic therapy remains unclear. Although rare, infectious complications have also been reported in neonates, raising concern (Cnota J et al., Pediatrics 2005; Kunz A N et al., J Pediatr Gastroenterol Nutr 2004). The current invention is aimed at providing new methods and compositions for the prevention and/or reduction of inflammation of the gastro-intestinal tract mucosal tissues. In particular the current invention aims at providing new methods and compositions for preventing and/or treating necrotizing enterocolitis in infants, preferably in preterm infants.

### BRIEF DESCRIPTION OF THE DRAWINGS

FIG. 1 is a schematic and cartoon explaining the model of the current invention in three stages, 1. Mucosa in healthy condition, with normal AP histochemistry and LPS detoxification. 2. Diseased condition, deficient AP staining, insufficient detoxification of LPS by AP, influx/translocation of toxic LPS from the gut into circulation leading to an inflammatory response 3. Restoration of mucosal AP levels and detoxification of LPS by providing an exogenous source of AP.

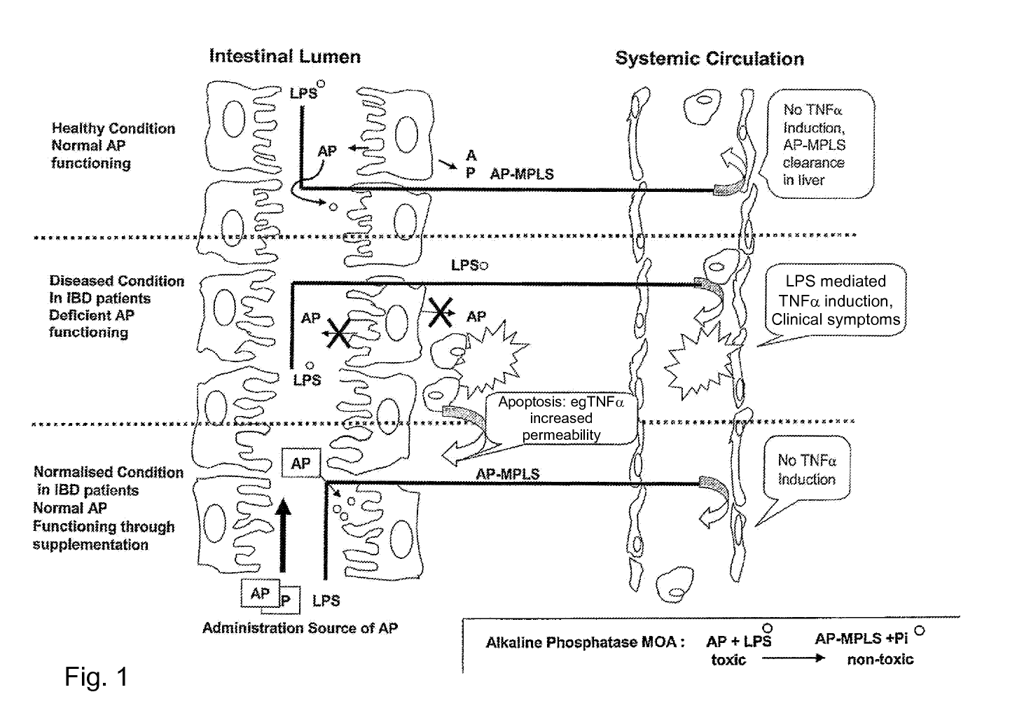

FIG. 2: Case fatality rate in mice with TNBS-induced colitis

FIG. 3: Case fatality rate in mice with Dextran Sulfate Sodium (DSS)-induced colitis

FIG. 4: Weight loss in AP-treated and non-treated mice with TNBS-induced colitis.

FIG. 5: Weight loss in AP treated and non treated animal with DSS-induced colitis

FIG. 6: Cytokine production in the colon of mice with TNBS-colitis. (a) The Th1 response in the colon; (b) the Th2 response in the colon

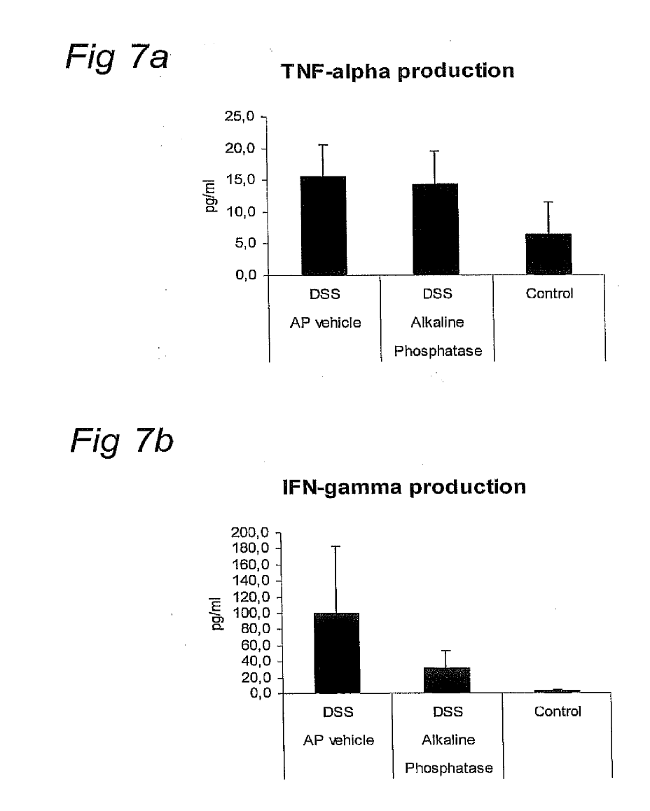

FIG. 7: Concentrations of (a) TNF-α and (b) IFN-γ in colon homogenates. The control mice are normal values of non-ill and unreated mice.

FIG. 8: DSS treatment decreases body weight. Mice were treated either with normal drinking water or with drinking water containing 2% DSS for 5 days. On day six, DSS treated animals had lost around 10% bodyweight compared with control animals and were defined “colitic.” Shown are mean with standard deviations of 29 animals per group.

FIG. 9: DSS treated mice consume between 40-80 mg DSS per day. Mice were treated either with drinking water or drinking water containing 2% DSS. Drinking bottles were weighed before and after refilling. Values show mean water intake in g/mouse/day.

FIG. 10: Alkaline phosphatase levels in different parts of intestinal tract at different time points after gavage. At different time points after gavage, mice were sacrificed and intestines removed. Feces from different parts of the tract were collected and alkaline phosphatase content measured. Shown are mean values of three mice per time point.

FIG. 11: Representative Western blot from a single experiment shows band densities for AP in adult duodenum, control, NEC, and iAP-treated intestines (molecular weight−65 kDa). p-Actin is shown to confirm equal protein loading (molecular weight=43 kDa). Densitometry data (average pixels) for AP from multiple experiments are shown graphically. Error bars represent SEM and p-values <0.05 are indicated with an asterisk (*). A=adult duodenum, C=control, N1=NEC-1 (3 doses LPS/day), N2=NEC-2 (2 doses LPS/day), I1=iAP-1 iAP/LPS mixed in first feed), I2=iAP-2 (iAP given without LPS in first feed), +=iAP positive control.

FIG. 12: AP activity is reported as U/mg protein. Error bars represent SEM and p-values<0.05 are indicated with an asterisk (*).

FIG. 13: Terminal ileum sections from control (a), NEC (b), and IAP-treated (c) pups (magnification 20×) probed with anti-alkaline phosphatase antibody and labeled fluorescently with Alexa-Fluor 633 (red).

FIG. 14: Terminal ileum H&E sections from control (a), NEC (b), and IAP-treated (c) pups (magnification 20×).

FIG. 15: Histologic injury grading of terminal ileum sections from control, NEC-1, NEC-2, iAP-1, and iAP-2 pups. A 4-point grading scale was used to characterize intestinal injury (0=normal histology, 1=mild submucosal/lamina propria separation, 2=moderate submucosal/lamina propria separation and/or edema, 3=severe edema, submucosal separation and regional villous sloughing, 4=necrosis, loss of villi). Grading reported as mean score. Error bars represent SEM and p-values <0.05 are indicated with an asterisk (*).

### DESCRIPTION OF THE PREFERRED EMBODIMENTS

### Definitions

Necrotizing enterocolitis (NEC) is an acute inflammatory disease of the gastro-intestinal tract, primarily occurring in sick or premature newborn infants. It can cause the death (necrosis) of intestinal tissue and progress to blood poisoning (septicemia). Although controversial, it is believed that NEC follows from a bacterial infection of the gastro-intestinal tract mucosa and that endotoxins play a major role in the pathogenesis.

Endotoxins are part of the outer membrane of the cell wall of Gram-negative bacteria. Endotoxins are invariably associated with Gram-negative bacteria whether the organisms are pathogens or not. Although the term “endotoxin” is occasionally used to refer to any cell-associated bacterial toxin, it is properly reserved to refer to the lipopolysaccharide or LPS complex associated with the outer membrane of Gram-negative bacteria such as Escherichia (E. coli), Salmonella, Shigella, Pseudomonas (Ps. aeruginosa), Neisseria (N. meningitidis), Haemophilus (H. influenzae), Chlamydia (Chl. pneumoniae), Helicobacter (H. pylori) and other leading pathogens.

Lipopolysaccharides are complex amphiphilic molecules with a monomeric molecular weight of about 10 kDa, that vary widely in chemical composition both between and among bacterial species. LPS consists of three components or regions, Lipid A, an R oligosaccharide and an O polysaccharide. Lipid A contains the hydrophobic, membrane-anchoring region of LPS. Lipid A consists of a phosphorylated N-acetylglucosamine (NAG) dimer with 6 or 7 fatty acids (FA) attached. The Core (R) antigen or R oligosaccharide is attached to the 6 position of one NAG. The R antigen consists of a short chain of sugars. Two unusual sugars are usually present, heptose and 2-keto-3-deoxyoctonoic acid (KDO), in the core polysaccharide. KDO is unique and invariably present in LPS and so has been an indicator in assays for LPS (endotoxin). The 0 polysaccharide or O-antigen is attached to the R-oligosaccharide and is a repetitive glycan polymer of varying length.

With minor variations, the core polysaccharide and lipid A is common to all members of a bacterial genus (e.g., Salmonella), but it is structurally distinct in other genera of Gram-negative bacteria. Salmonella, Shigella and Escherichia have similar but not identical cores.

The biological activity of endotoxin is associated with the lipopolysaccharide (LPS). Toxicity is associated with the lipid component (Lipid A) and immunogenicity is associated with the polysaccharide components. The cell wall antigens (0 antigens) of Gram-negative bacteria are components of LPS. LPS elicits a variety of inflammatory responses in an animal. Because it activates complement by the alternative (properdin) pathway, it is often part of the pathology of Gram-negative bacterial infections.

The Limulus assay (LAL) is a well known bioassay in the art to measure LPS concentrations and toxicity. The assay is based on an exquisitely sensitive primitive defense system of the ancient horseshoe crab, Limulus polyphemus. An assay based on this system can be measured by a color change after cleavage of chromogenic or fluorogenic substrates. LAL can be used to measure sub-picogram quantities of these microbial products very rapidly with minimal equipment and can detect live, dead and non-cultivable organisms. The blood cells of Limulus, or amebocytes, of the horseshoe crab constitute a primitive “innate” immune defense, binding to the outer cell wall structures of the microbial cell and causing a blood clotting reaction. Soluble LPS, as well as cell wall components of other microbes, such as 0 glucans in yeast and fungi, have been shown to cause the horseshoe crab blood to clot. This clotting reaction is now known to be an enzyme cascade whose components are present in granules within the amebocyte. A lysate of the amebocyte is produced by collecting blood cells in a sterile, endotoxin-free method and is available as a commercial product (LAL, Charles River Endosafe, Charleston, S.C.) currently used as an assay for LPS and detoxification of LPS by AP enzymes and compositions comprising sources of AP.

Alkaline phosphatase (AP): EC 3.1.3.1 according to IUBMB Enzyme Nomenclature, the common name is alkaline phosphatase (AP), an enzyme that catalyzes the reaction of a phosphate monoester+H₂O→an alcohol+phosphate. Other name(s) for AP are alkaline phosphomonoesterase; phosphomonoesterase; glycerophosphatase; alkaline phosphohydrolase; alkaline phenyl phosphatase; orthophosphoric-monoester phosphohydrolase (alkaline optimum). The systematic name of AP is phosphate-monoester phosphohydrolase (alkaline optimum).

AP is a wide specificity enzyme, it also catalyses transphosphorylations. In humans and other mammals, at least four distinct but related alkaline phosphatases are known. They are intestinal, placental, placental-like, and liver/bone/kidney (or tissue non-specific) alkaline phosphatase. The first three are located together on chromosome 2 while the tissue non-specific form is located on chromosome 1. The exact physiological functions of the APs are not known, but AP appears to be involved with a large number of physiological processes, among which the detoxification of LPS through dephosphorylation of the toxicity determining lipid A moiety of LPS. For the current invention, the term alkaline phosphatase may comprise any enzyme exhibiting detoxification of LPS as determined by a Limulus assay or another bioassay. The activity of an AP enzyme or composition or preparation comprising AP can be determined by detoxification of commercially available LPS (for instance Lipopolysaccharide (LPS) from Sigma, Cat. No. L-8274) in vitro, followed by a standard Limulus assay (LAL) before and after AP treatment. Alternatively LPS toxicity reduction through AP activity can be quantitated by means of a bioassay as described by Beumer et al., 2003.

Mucosa is a mucus-secreting membrane lining all body cavities or passages that communicate with the exterior. Mucosa is a moist tissue that lines many organs (such as the intestines) and body cavities (such as nose, mouth, lungs, vagina, bile duct, esophagus) and secretes mucous (a thick fluid). The mucosa, or mucous membrane, is a type of tissue that protects body cavities from environmental conditions, pathogens and toxic substances and are usually moist tissues that are bathed by secretions (such as secretions in the bowel, lung, nose, mouth and vagina).

The current invention is aimed at providing new methods and compositions for the detoxification of LPS in situ at mucosal tissues in body cavities. A first aim of the in situ detoxification of LPS at mucosal surfaces in the body is to prevent or reduce local inflammatory response at such surfaces, in particular at the gastro-intestinal tract mucosal surface. Furthermore, the LPS that is thus detoxified is no longer available for passage through mucosal layers and thus cannot enter the circulation where it will exert its toxic effects and/or cause a further local and/or systemic inflammatory response. Detoxification may also comprise neutralizing or complexation of LPS by AP, which by close proximity may form a detoxified composition. The methods comprise the use of sources of alkaline phosphatase, which is known to be a potent means for LPS detoxification. A source of AP can be any AP enzyme, or any composition comprising the AP enzyme and any means which is capable of producing a functional AP enzyme in the context of the current invention, such as DNA or RNA nucleic acids encoding an AP enzyme. The nucleic acid encoding AP may be embedded in suitable vectors such as plasmids, phagemids, phages, (retro)viruses, transposons, gene therapy vectors and other vectors capable of inducing or conferring production of AP. Also native or recombinant micro-organisms, such as bacteria, fungi, protozoa and yeast may be applied as a source of AP in the context of the current invention.

In a first embodiment, the invention provides a medical food, preferably an infant formula or a (human) milk fortifier, comprising enzymatically active alkaline phosphatase (AP) or an enzymatically active part thereof. A medical food according to the invention, preferably an infant formula or (human) milk fortifier according to the invention is for instance suitable for preventing or reducing inflammation of the gastro-intestinal mucosa, such as for instance seen in necrotizing enterocolitis, when the AP is delivered to the mucosa of the gastro-intestinal tract. A medical food, preferably an infant formula or milk fortifier for preventing or reducing inflammation of the gastro-intestinal mucosa is especially useful, because it can be easily administered to a subject in addition to, or (partly) replacing a commercially available infant formulas devoid of enzymatically active alkaline phosphatase. However, a medical food according to the invention, preferably an infant formula or fortifier according to the invention can also be administered in addition to, or partly replacing the commercially available infant formulas devoid of enzymatically active alkaline phosphatase. With partly replacing is meant that for instance only 1 or more of the infant formula feedings per day is replaced by an a medical food according to the invention, preferably infant formula or fortifier according to the invention. It is also possible to replace only part of a commercial available infant formula, for instance only 1 or more spoonfuls per feeding with a medical food, formula or fortifier according to the invention. A medical food, infant formula or (human) milk fortifier can also be administered in addition to the infant formula or mother's milk.

Medical foods are foods that are specially formulated and intended for the dietary management of a disease that has distinctive nutritional needs that cannot be met by normal diet alone. They were defined in the Food and Drug Administration's 1988 Orphan Drug Act Amendments and are subject to the general food and safety labeling requirements of the Federal Food, Drug, and Cosmetic Act (see for instance the World Wide web URL of fda.gov/Food/GuidanceComplianceRegulatoryInformation/GuidanceDocuments/MedicalFoods/UC.

Medical foods are distinct from the broader category of foods for special dietary use and from traditional foods that bear a health claim. In order to be considered a medical food the product must, at a minimum, be:
a food for oral ingestion or tube feeding (nasogastric tube)
labeled for the dietary management of a specific medical disorder, disease or condition for which there are distinctive nutritional requirements, and
intended to be used under medical supervision.
Medical foods can generally be classified into the following categories:
Nutritionally complete formulas
Nutritionally incomplete formulas
Formulas for metabolic disorders
Oral rehydration products

According to the U.S. Orphan Drug Act (21 U.S.C. §360 ee (b)(3)), a medical food is defined as a food formulated to be consumed or administered enterally under the supervision of a physician and which is intended for the specific dietary management of a disease or condition for which distinctive nutritional requirements, based on recognized scientific principles, are established by medical evaluation.

An infant formula is defined herewith as a formula in compliance with nutritional requirements for infants. An infant is herewith defined as a preterm or term baby, not older than 6 months, preferably not older than 3 months. In the US, the Food and Drug Administration (FDA) oversees the manufacture of infant formulas and has set minimum requirements with regard to nutrient levels (§412. of 21 USC §350(a) Requirements for Infant Formulas). According to the FDA, an infant formula shall contain nutrients in accordance with Table 1, or, if revised by the Secretary), as so revised.

TABLE 1
nutrients in infant formula prescribed by the FDA

| Nutrient | Minimum¹ | Maximum¹ |
|---|---|---|
| Protein (gm) | 1.8² | 4.5. |
| Fat: | | |
| gm | 3.3 | 6.0. |
| percent cal | 30.0 | 54.0. |
| Essential fatty acids | | |
| (linoleate): | | |
| percent cal. | 2.7 | |
| mg | 300.0 | |
| Vitamins: | | |
| A (IU) | 250.0 (75 μg)³ | 750.0 (225 μg)³ |
| D (IU) | 40.0 | 100.0. |
| K (μg) | 4.0 | |
| E (IU) | 0.7 (with 0.7 IU/gm | |
| | linoleic acid). | |
| C (ascorbic acid) (mg) | 8.0 | |
| B1 (thiamine) (μg) | 40.0 | |
| B2 (riboflavin) (μg) | 60.0 | |
| B6 (pyridoxine) (μg) | 35.0 (with 15 μg/gm of | |
| | protein in formula). | |
| B12 (μg) | 0.15 | |
| Niacin (μg) | 250.0 | |
| Folic acid (μg) | 4.0 | |
| Pantothenic acid (μg) | 300.0 | |
| Biotin (μg) | 1.5⁴ | |
| Choline (mg) | 7.0⁴ | |
| Inositol (mg) | 4.0⁴ | |
| Minerals: | | |
| Calcium (mg) | 50.0⁵ | |
| Phosphorus (mg) | 25.0⁵ | |
| Magnesium (mg) | 6.0 | |
| Iron (mg) | 0.15 | |
| Iodine (μg) | 5.0 | |
| Zinc (mg) | 0.5 | |
| Copper (μg) | 60.0 | |
| Manganese (μg) | 5.0 | |
| Sodium (mg) | 20.0 | 60.0. |
| Potassium (mg) | 80.0 | 200.0. |
| Chloride (mg) | 55.0 | 150.0. |

¹Stated per 100 kilocalories.
²The source of protein shall be at least nutritionally equivalent to casein.
³Retinol equivalents.
⁴Required to be included in this amount only in formulas which are not milk-based.
⁵Calcium to phosphorus ratio must be no less than 1.1 nor more than 2.0.

In addition, formula manufacturers set nutrient levels that are generally above the FDA minimum requirements. Furthermore, the FDA exempts any infant formula which is represented and labeled for use by an infant who has an inborn error of metabolism or a low birth weight, or who otherwise has an unusual medical or dietary problem from the nutritional requirements as laid down in Table I. It is therefore that an infant formula according to the invention is preferably in accordance with the FDA regulation §412. [21 USC §350a], but that at least some of the nutrients of the infant formula according to the invention may be outside the specification of Table I.

Most infant formulas are based on cow milk. However, some infants react allergic to proteins contained in cow milk. Such reactions or the risk of such reaction is lower when cow milk is (partially) hydrolyzed in order to destroy the immunogenic protein fragments that cause these allergic reactions. During hydrolysis, the large proteins are chopped in to smaller pieces that are less immunogenic. The use of soy protein instead of cow milk decreases the risk of cow milk allergy, but as a consequence, the infant may develop a soy allergy.

Human milk fortifiers are a special kind of infant formula, intended to be mixed with human breast milk. Human milk fortifier helps to enrich the calorie, fat and protein content of human milk, especially when feeding pre-term and/or low-birthweight babies. Human milk fortifier is mainly used for premature infants, but other low birth weight infants may also benefit from human milk fortifier. A human milk fortifier adds extra calories and nutrients to breast milk. Most human milk fortifiers are derived from typical cow's milk, but some varieties are derived from soy and/or are made to be hypoallergenic and lactose-free. In addition, a (human) milk fortifier according to the invention comprises enzymatically active alkaline phosphatase. A (human) milk fortifier according to the invention may also be mixed in with an infant formula to add extra calories to said formula.

In a preferred embodiment, a medical food according to the invention is provided, wherein the medical food comprises a dry powder milk, a dry powder milk hydrolysate, or dry powder soy protein. In another preferred embodiment, the medical food is a liquid formulation, preferably based on (cow) milk, optionally (partly) hydrolyzed to minimize the risk of allergy to cow milk protein, or based on soy protein. It is preferred that the medical food is an infant formula or (human) milk fortifier.

With the term “dry powder” is meant that the moisture content is preferably less than 5%, more preferably less than 4%. Of course the moisture content can also be lower. Reduction of moisture content has several advantages, such as for instance reduced weight of the packaged infant formula. Reduced moisture content, however also inhibits microbial growth in the final product. Although the dry powder medical food according to the invention is preferably sterile when packaged, it will generally not remain sterile during usage, because typically for every feeding the box is opened in a non-sterile environment. It is therefore that in a preferred embodiment, the dry powder milk, the dry powder milk hydrolysate, or the dry powder soy protein of the invention comprises less than 5%, more preferably less than 4%, more preferably less than 3% moisture.

Currently known, commercially available powdered infant formula is manufactured by more than a dozen companies in 40-50 processing plants worldwide. The manufacturing processes are very similar, wherein two general types of processes are commonly used: a dry blending process and a wet mixing—spray drying process, or a combination thereof. In the combined process, a base powder (consisting mainly of protein and fat components) is produced using the wet mixing and spray drying process and then the base powder is dry blended with carbohydrate, mineral and vitamin ingredients. These processes have different risks and benefits with respect to the potential for product contamination by microorganisms.

### Dry Blending

In the dry blending process, the ingredients necessary for a complete infant formula product are mixed together in a dehydrated powdered form. As already explained above, the moisture content of dehydrated powdered ingredients must not necessarily be 0%, but is preferably less than 5% in order to reduce the risk of microbial growth. Dry blending, including combined wet and dry processes, offers some manufacturing advantages over wet blending—spray drying. Dry blending is less capital intensive and more energy efficient than wet mixing—spray drying. Also, since dry blending does not involve the use of water in the manufacturing process, the processing line can be kept dry for long periods of time. In a dry environment, the chance that harmful bacteria will become established in the plant environment in sufficient numbers to cause product contamination is reduced. However, as there is no heat treatment to destroy bacteria in the final product, the microbiological quality of a dry-blended product is largely determined by the microbiological quality of the constituent dry ingredients. Thus, bacteria present in one of the supplied ingredients are likely to be present in the finished product. Although the ingredients are typically tested for, microbial contamination, it is difficult to assure microbiological quality by lot testing alone. Therefore the ingredients are generally obtained from suppliers that adhere to good manufacturing practices.

Dry ingredients are normally blended in large batches (1,000 to 5,000 lbs.) in a ribbon blender or other large scale blending equipment. The ingredients must be blended until the nutrients are uniformly distributed throughout the batch. The blended product is then passed through a sifter to remove oversize particles and extraneous material. The sifted product is then transferred to, e.g., bags, totes or lined fiberboard drums for storage. In some cases, the powder is transferred directly to a powder packaging line. At the packaging line, the powder is for instance transferred to a filler hopper that feeds powder into a can filling line. Filled cans are typically flushed with inert gas, seamed, labeled, coded and packed into cartons. Typically, finished product is held until it undergoes a final check for conformance to specifications, including testing for microbiological contaminants.

### Wet Blending—Spray Drying

In the wet blending—spray drying process, ingredients are typically blended together, homogenized, pasteurized and spray dried to produce a powdered product. A pasteurization step generally destroys harmful bacteria that may be present in the ingredients, making the process much less dependent on the microbiological quality of ingredients. This process also has an advantage that nutrients are more uniformly distributed throughout the batch. However, the wet blending step and required cleaning of the process equipment provides the moisture needed by bacteria to grow and become established in the plant environment. Manufacturers that use the wet blending—spray drying process typically partition their plants into wet process and dry process areas and strictly limit the movement of people and equipment between the wet and dry areas. Also, they strictly enforce sanitation practices, particularly in the dry processing areas.

As with the dry blending process, ingredients are generally tested for conformance to specifications. Ingredients are typically blended with water in large batches (1,000-5,000 gallons) and thereafter pasteurized and homogenized. The severity of the pasteurization process varies among manufacturers, but it is always sufficient to destroy the vegetative cells of harmful bacteria. After pasteurization, certain heat sensitive micro nutrients (e.g., vitamins, amino acids and fatty acids) are added to the product. The microbiological quality of these nutrients is critical, since the product may not receive further heating sufficient to destroy harmful bacteria as such heating would also destroy the heat sensitive micro nutrients. After the addition of micro-nutrients, the liquid may be concentrated by passing it through an evaporator or it may be pumped directly to a spray dryer. If the product is evaporated, it is typically heated to 145-170° F. (63° C.-77° C.) during the process. In some cases, the concentrated liquid may be cooled to less than 45° F. (7° C.) and stored in a large tank (10,000-30,000 gallons) until needed. Prior to spray drying, the product is often pre-heated to 160-200° F. (71-93° C.) and passed through a high pressure pump to spray dryer nozzles. The product is atomized into the spray dryer where the inlet air temperature typically ranges from 280-400° F. (138-204° C.), depending on the dryer model and process requirements. The spray dryer may be a horizontal box-type dryer or a vertical funnel shaped dryer or any other dryer known in the art. As the droplets of product pass through the dryer, water is evaporated and the dry powder falls to the bottom of the spray dryer. The outlet temperature of the dryer is normally approximately 220° F. (104° C.). The temperature of the powder at the spray dryer outlet typically ranges from 110-175° F. (43-79° C.). The warm powder is often passed through a fluidized bed where it is cooled by a stream of chilled air. At the end of the fluidized bed, the powder has cooled to approximately 70° F. (21° C.). Typically, HEPA-filtered air is supplied to the spray dryer and fluidized bed to minimize the risk of product contamination. After spray drying, the product may be agglomerated to increase the particle size and to improve its solubility. The sifted product is then transferred to for instance bags, totes or lined fiberboard drums for storage. In some cases, the powder is transferred directly to a powder packaging line. At the packaging line, the powder is for instance transferred to a filler hopper that feeds powder into a can filling line. Filled cans are typically flushed with inert gas, seamed, labeled, coded and packed into cartons. Typically, finished product is held until it undergoes a final check for conformance to specifications, including testing for microbiological contaminants.

One major drawback of the commercially available infant formulas or (human) milk fortifiers is that during the process of manufacture, several heating steps are necessary for either controlling microbial contaminants, or for instance for spray drying the liquid formula into a dry powder form. During these processes, a natural ingredient of for instance cow milk, alkaline phosphatase, is destroyed, at least concerning its enzymatic activity. The present invention, however, shows that the enzymatically active alkaline phosphatase is able to prevent or reduce inflammation of the gastro-intestinal tract. The high incidence of NEC in premature, non-breast-fed infants can be explained by the lack of alkaline phosphatase enzymatic activity in infant formula generally used to replace the mother's milk. It is therefore that a medical food, infant formula or milk fortifier according to the invention is especially useful in preventing and/or reducing NEC in infants. In a preferred embodiment, therefore a medical food according to the invention is provided, wherein the medical food is designed to treat or prevent NEC

Although commercially available human milk fortifiers are generally used for mixing in with human milk, in one aspect, the invention provides a medical food (e.g., a milk fortifier) comprising enzymatically active alkaline phosphatase to be mixed in with a commercially available infant formula. In another aspect, the invention provides a medical food comprising enzymatically active alkaline phosphatase to be mixed with human milk. The human milk can be from the mother, but it is also possible to use (commercially available) donor milk. Generally, human donor milk is pasteurized or sterilized in order to destroy pathogenic microorganisms or in order to prolong storability. As said above, the pasteurization or sterilization step normally destroys enzymatic activity of the alkaline phosphatase enzyme. A medical food according to the invention, comprising enzymatically active alkaline phosphatase, is thus especially useful for the prevention and/or treatment of NEC when human donor milk is used for nutrition of an infant. It is preferred that such medical food is formulated as a (human) milk fortifier.

An infant formula or (human) milk fortifier according to the present invention can be prepared according to methods known in the art, for instance as described above, provided the final infant formula or fortifier comprises enzymatically active alkaline phosphatase in an amount suitable for preventing and/or reducing inflammation of the gastro-intestinal tract mucosa, when the infant formula or fortifier is administered to said mucosa. Cow milk already contains alkaline phosphatase, but during the manufacturing steps of cow-milk-based infant formula or fortifier, the enzyme is inactivated, due to for instance the heating steps that are generally used to prevent microbial spoilage. One method for preparing an infant formula or fortifier according to the invention is thus to prevent enzymatic inactivation of the enzymatically active alkaline phosphatase present in cow milk used for the preparation of infant formula or fortifier. This can for instance be achieved by sterile filtration of the (ingredient of the) infant formula or fortifier instead of pasteurization or sterilization. It is also possible to add a suitable amount of enzymatically active alkaline phosphatase to the product after the heating step(s).

Most natural alkaline phosphatase isoenzymes are heat instable, meaning that they generally lose their enzymatic activity during prolonged heating above approximately 160° F. (71° C.). Hence, in order to prepare a medical food, infant formula or fortifier according to the invention with enzymatically active alkaline phosphatase, it is important that once the enzymatically active alkaline phosphatase is present (either added during the manufacturing process or already contained in one of the ingredients, for instance cow milk), the heating of the medical food, infant formula or fortifier preparation is kept to a minimum. Preferably alkaline phosphatase is added after pasteurization and/or spray drying of the medical food, infant formula or fortifier preparation. Alternatively a heat-stable alkaline phosphatase, such as human placental alkaline phosphatase or for instance a heat-stable recombinant alkaline phosphatase is used for a medical food, infant formula or fortifier of the invention.

Commercially available liquid infant formula is generally sterilized in order to prolong storability of the final product. Sterilization generally takes place at higher temperatures (210-250° F./99-121° C.) than pasteurization. In such case, alkaline phosphatase is preferably added as a sterile solution to the infant formula after the last heating step and prior to sterile filling of the product packages.

In a preferred embodiment, a medial food, infant formula or fortifier according to the invention is provided, wherein the AP is a mammalian intestinal AP, a tissue non specific AP, a placental AP or a liver AP. More preferably, the AP is of human or bovine origin. Instead of isolating a natural occurring AP, such as for instance present in milk, it is also possible to add a recombinantly expressed enzymatically active AP to a medical food, infant formula or fortifier according to the invention. A recombinantly expressed AP can be the whole AP protein of a naturally occurring isoform, but it is also possible to express and use a modified recombinant phosphatase. Such modification may comprise for instance a single amino acid substation, but it is also possible to recombinantly express for instance a chimeric protein. Such modified APs are extensively described in U.S. patent application Ser. No. 12/451,137, which is herewith incorporated by reference. A recombinant modified alkaline phosphatase may also have increased heat stability, such that it can be added prior to for instance pasteurization of the infant formula mixture. Also a minimal enzyme is provided, such as for instance a polypeptide comprising at least the catalytic domain of an AP. In a preferred embodiment, therefore, a medical food according to the invention is provided comprising an enzymatically active recombinant AP or a modified recombinant AP, or an enzymatically active part thereof. Preferably said modified recombinant AP consists of a crown domain of one AP and a catalytic domain of another AP. More preferably said modified recombinant AP consists of a crown domain of human placental AP and a catalytic domain of human intestinal AP. The medical food is preferably formulated as an infant formula or a (human) milk fortifier.

The AP present in a medical food according to the invention can be obtained using a wide variety of methods known in the art. The AP can for instance be isolated and purified from a natural source. A commercial source of intestinal AP is for instance AP isolated and purified from bovine calf intestines. AP can also be isolated and purified from a food product, such as milk. Another possibility to obtain AP for use in a medical food according to the invention is recombinant expression. Recombinant expression of AP can for instance be achieved in an in vitro system, such as yeast or (human) cell culture, in plants, such as for instance a tobacco plant, or in vivo, such as for instance the expression of a recombinant AP in cows. After expression of the recombinant AP, the AP is preferably isolated and purified. This can be achieved by methods known in the art.

In a preferred embodiment, the medical food according to the invention comprises AP that is enteric coated for oral administration and delivery to the gastro-intestinal tract mucosa. Such enteric coated AP is less susceptible to for instance moisture during storage and less susceptible to the acidic environment in the stomach. Many of the naturally occurring AP isoforms are acid-labile, i.e. low pH irreversibly inactivates the enzymatic activity of AP. Up to the present invention it was thought that in order for AP to be active in the intestinal tract, it had to be protected to pass the acidic environment in the stomach. In a working example in rat pups, however, the inventors have surprisingly shown that non-coated AP given orally to the pups was able to rescue them from NEC. In another preferred embodiment, therefore, a medical food according to the invention is provided, wherein the enzymatically active AP is not enteric coated. In a more preferred embodiment, the medical food is formulated as an infant formula or a (human) milk fortifier.

In a working example, the present inventors have shown that oral gavage of around 5000 IU of AP/kg/day could reduce the severity of ulcerative colitis in a mouse model. In another working example, the present inventors have shown that rat pups at risk of necrotizing enterocolitis can be successfully treated by a gavage of around 800 IU of AP/kg/day. Considering the dosages in the NEC rat model, this would amount to approximately a daily dose of around 1500 IU of AP for a 2000 g infant. For a 600 g (preterm) infant this would amount to approximately 500 IU per day. The daily dose may be given in one feed, but it is also possible to spread out the daily dose over several feedings, for instance 6× per day 100 IU AP. The optimal dosing is thus around 500 IU per day but can be lower or higher. The most efficient dosing will be determined in a controlled, clinical efficacy study.

In a preferred embodiment, the invention provides an infant formula according to the invention, comprising between 0.05 and 50,000 IU AP/g dry powder, preferably between 0.5 and 5,000 IU AP/g of dry powder. More preferably the infant formula comprises between 5 and 500 IU AP/g of dry powder, more preferably between 20 and 200 IU AP/g of dry powder, most preferably between 40 and 100 IU AP/g of dry powder. It is of course also possible to provide an infant formula according to the invention comprising more than 50,000 IU/g of dry powder and mix the infant formula of the invention for instance with a commercially available infant formula in order to optimize the dosage of alkaline phosphatase in the infant formula.

A (human) milk fortifier is generally given in lower quantities, because it is generally mixed in with human milk or with an infant formula. A milk fortifier according to the invention preferably comprises between 0.2 and 200,000 IU AP/g of dry powder. More preferably the fortifier comprises between 2 and 20,000 IU AP/g of dry powder, more preferably between 20 and 2,000 IU AP/g of dry powder, even more preferably between 80 and 800 IU AP/g of dry powder, most preferably between 160 and 400 IU AP/g of dry powder. It is also possible to use more than 200,000 IU AP/g of dry powder and use less powder.

A liquid infant formula according to the invention preferably comprises between 0.8 and 800,000 IU AP/liter, more preferably between 8 and 80,000 IU/liter, more preferably between 80 and 8,000 IU AP/liter, even more preferably between 300 and 3,000 IU AP/liter, most preferably between 600 and 1,500 IU AP/liter of liquid formulation. The liquid (human) milk fortifier according to the invention preferably comprises between 3 and 3,000,000 IU AP/liter, more preferably between 30 and 300,000 IU AP/liter, more preferably between 300 and 30,000 IU AP/liter, even more preferably between 1200 and 7,500 IU AP/liter, most preferably between 2,500 and 4,000 IU AP/liter. Of course, the liquid formulas according to the invention may also comprise more than 800,000 IU AP/g dry powder or more than 3,000,000 IU AP/liter.

It is thus possible to prevent or reduce necrotizing enterocolitis in a subject by administering to the mucosal layer of the gastro-intestinal tract of said subject an enzymatically active alkaline phosphatase. In one embodiment therefore, the invention provides a method for preventing and/or reducing necrotizing colitis in a subject, comprising administering to the mucosal layer of the gastrointestinal tract of said subject an enzymatically active alkaline phosphatase.

In a preferred embodiment, a method according to the invention is provided, wherein said alkaline phosphatase is administered orally. In another preferred embodiment, the alkaline phosphatase is administered as a suppository or as a duodenal drip. Parenteral administration, i.e. administered by means other than through the alimentary tract (as by intramuscular or intravenous injection) is explicitly excluded as a method for administering AP to the mucosal layer of the gastro-intestinal tract.

In another preferred embodiment, said alkaline phosphatase is administered at least once per day, more preferably at least twice per day, more preferably at least three times per day, more preferably at least four times per day, more preferably at least five times per day, most preferably at least six times or more per day. Spreading of the dosages over the day has the advantage that alkaline phosphatase is continuously present in the intestinal tract of a subject receiving enzymatically active AP is. This may lead to improved effectiveness of the enzymatically active AP on the prevention or reduction of NEC.

In a preferred embodiment, the subject is an infant, i.e. a preterm or term infant not older than 3 months. In humans, preterm birth refers to the birth of a baby of less than 37 weeks gestational age. As a consequence a term (or full term) birth refers to the birth of a baby of more than or equal to 37 weeks gestational age.

Especially in infants, it is preferred to administer a medical food according to the invention, more preferably an infant formula or fortifier according to the present invention. A subject preferably receives an enzymatically active alkaline phosphatase of the invention when at least one of the risk factors for developing necrotizing enterocolitis is present in said subject. These are, amongst others, preterm birth, hypoxia before, during or after birth, or deprivation of breast feeding. Also subjects presenting with at least one sign and/or symptom of necrotizing enterocolitis are preferably treated with a method according to the invention.

Initial symptoms of NEC vary and may include feeding intolerance, abdominal distension, bloody stools, apnea, lethargy, temperature instability or hypoperfusion. Classically, increased amounts of gastric residual and abdominal distension are noted. A careful and meticulous abdominal exam might elicit localized tenderness.

Diagnosis of NEC is based on clinical symptoms and radiology. The abdominal X-ray is the best diagnostic tool in the evaluation of NEC. However, there is a wide range of inter-observer variability in its interpretation. Pneumatosis intestinalis (air in the bowel wall), when present, is diagnostic of NEC. It is thought to be due to the production of gas from bacterial fermentation. X-ray might also show a) Pneumatosis intestinalis (diagnostic of NEC) and b) Pneumoperitoneum (free air in the peritoneal cavity) and c) Non specific radiological findings include a persistently abnormal gas pattern, thickened loops, ascites or a gasless abdomen.

Bell and coworkers have proposed a clinical staging classification to describe NEC severity:

### Stage I—Suspected NEC:

Systemic: Non-specific signs such as temperature instability, lethargy, apnea, bradycardia

GIT: Gastric residuals, occult blood in stool

AXR: Normal/non-specific changes

### Stage IIA—Definite and Mild NEC;

Systemic: Non-specific signs

GIT: Abdominal distension±tenderness, absent bowel sounds, frank blood in stool

AXR: Ileus, focal pneumatosis intestinalis

### Stage IIB—Definite and Moderate NEC

Systemic: Mild acidosis, thrombocytopenia

GIT: Abdominal wall edema, tenderness±mass

AXR: Extensive pneumatosis intestinalis, early ascites, ±intrahepatic portal gas

### Stage IIIA—Advanced NEC

Systemic: Respiratory/metabolic acidosis, apnea, hypotension, decreasing urine output, leukopenia, DIC

GIT: Spreading edema, erythema, induration of the abdomen

AXR: Prominent ascites±persistent sentinel loop, no perforation

### Stage IIIB—Advanced NEC

Systemic: Deteriorating vital signs, shock, electrolyte imbalance

GIT and AXR: Signs of perforation

In one embodiment, the invention thus provides a method for preventing and/or treating necrotizing enterocolitis in a subject, comprising administering to the mucosal layer of the gastro-intestinal tract of the subject an enzymatically active alkaline phosphatase.

In a preferred embodiment, a method according to the invention is provided, wherein the subject is a preterm or term infant not older than 3 months of age. In an even more preferred embodiment, the subject is a preterm, infant. In another more preferred embodiment, the preterm or term infant is born with a low birthweight (<2.5 kg), more preferably with a very low birthweight (<1.5 kg), most preferably with an extremely low birthweight (<1.0 kg). The term “preterm infant” includes near term infants (born at <37 but >33 weeks of gestational age), moderate pre-term infants (<33 but >31 weeks of gestational age), severe preterm infants (<31 but >28 weeks of gestational age) and extremely preterm infants (<28 weeks of gestational age).

The enzymatically active AP can be administered to the mucosal layer of the gastro-intestinal tract by methods known in the art. Preferred methods are oral administration or by tube feeding, for instance via gastric tube, transpyloric tube. But also rectal administration, for instance via a suppository or a rectal tube, is possible.

It is also possible to use a commercially available infant formula, or even mother's milk, and add a suitable amount of enzymatically active alkaline phosphatase thereto. This can for instance be done by adding a suitable amount of enzymatically active alkaline phosphatase to a commercially available dry powder infant formula. It is also possible to add a suitable amount of enzymatically active alkaline phosphatase to a liquid infant formula or to mother's milk to obtain an infant formula for use in a method according to the invention.

With a suitable amount of enzymatically active alkaline phosphatase is meant that the amount of alkaline phosphatase is capable of preventing and/or reducing inflammation of the gastro-intestinal tract when the infant formula is administered to the mucosal layer of the gastrointestinal tract, preferably orally. A suitable amount of alkaline phosphatase is preferably between 0.8 and 800,000 IU/kg bodyweight. More preferably the amount is between 8 and 80,000 IU/kg bodyweight, more preferably between 80 and 8,000 IU/kg bodyweight, even more preferably between 400 and 2,000 IU/kg bodyweight, most preferably between 600 and 1,000 IU/kg bodyweight.

The invention thus provides a method for manufacturing an infant formula or a (human) milk fortifier for use in preventing and/or treating NEC, the method comprising the steps of: adding a suitable amount of enzymatically active alkaline phosphatase to a dry powder infant formula, to a dry powder (human) milk fortifier, to a liquid infant formula, to a liquid (human) milk fortifier, and/or to human milk.

An infant formula or fortifier prepared by a method according to the invention can be used instantly, but it is also possible to store the infant formula or fortifier for later use. In the latter case, however, the manufacturing steps must be carried out essentially aseptically or sterile, or the final product is preferably sterilized, for instance by filtration or by radiation, or refrigerated in order to prevent and/or reduce microbial spoilage.

In another embodiment the invention provides a method for the prevention or reduction of toxicity of LPS at a mucosal lining of a mammalian body cavity comprising the step of administering a source of AP at the mucosal layer. For those jurisdictions where methods of treatment are unpatentable by law, the invention likewise pertains to the use of AP as defined above, or the use of a composition containing a source of alkaline phosphatase as defined above. The source of AP is used for the manufacture of a medicament for delivery of AP at a mucosal layer for the prevention or reduction of toxic LPS influx through a mucosal lining of a mammalian body cavity. In an additional embodiment the invention provides a method for the prevention or reduction of toxic LPS influx through a mucosal lining of a mammalian body cavity comprising the step of administering a source of AP at the mucosal layer.

In particular the above mentioned method of administering a source of AP at mucosal layers of body cavities is suited for the treatment or prophylaxis of LPS mediated or exacerbated diseases, although the method may also be advantageously used for healthy subjects as a prophylactic treatment aimed at the prevention of LPS induced toxicity and/or LPS induced or exacerbated diseases. The beneficial effects of AP administration to reduce toxic LPS levels in body cavities and at mucosal layers according to the current invention will generate a general health promoting effect regardless of the medical condition of the subject treated. The health promoting effect may be further augmented by the consequent decrease in LPS influx through mucosal layers. An LPS mediated or induced disease may be any disease, symptom or group of symptoms caused by LPS toxicity. An LPS exacerbated disease may be any disease or symptom that is not directly caused by LPS or LPS toxicity but a disease which symptoms and clinical features may be aggravated by LPS and the clinical state of the subject suffering from such a disease is worsened by LPS and LPS toxicity.

Preferably the method is aimed at the treatment of an LPS mediated or exacerbated diseases selected from the group consisting of: inflammatory bowel diseases, sepsis/septic shock, systemic inflammatory response syndrome (SIRS), Meningococcemia, trauma/hemorrhagic shock, burn injuries, cardiovascular surgery/cardiopulmonary bypass, liver surgery/transplant, liver disease, pancreatitis, (necrotizing) enterocolitis, periodontal disease, pneumonia, cystic fibrosis, asthma, coronary heart disease, congestive heart failure, renal disease, hemolytic uremic syndrome, kidney dialysis, autoimmune diseases, cancer, Alzheimer, rheumatoid arthritis, lupus, systemic lupus erythematosus.

Circulating endotoxin has been detected in patients with inflammatory bowel diseases, in particular in patients diagnosed with Crohn's disease and ulcerative colitis. Its presence is the consequence of the damaged intestinal mucosa and increased LPS influx or gut translocation and causes or exacerbates the inflammatory response in the intestines. Intestinal bacterial translocation and LPS gut translocation is also observed in acute pancreatitis and liver diseases caused by cirrhosis, alcohol abuse, obstructive jaundice and other hepatic conditions. Endotoxin has also been implicated in the development of periodontal disease, where it penetrates the gingival epithelium/mucosa, ensuing a local inflammatory response. In a preferred embodiment the method comprises oral administration of a source of AP to reduce LPS toxicity at and/or passage of LPS through the mucosa.

One mode of administration comprises the use of pharmaceutical compositions comprising sources of AP, which may be delivered in a daily doses regimen to reduce toxic LPS levels in the lumen of the GI tract for a prolonged period of time. Preferably the pharmaceutical compositions comprise an enteric coating to protect AP from the detrimental effects of gastric juices (pH 1.0 to 2.5) and ensure efficient delivery of AP at the mucosa of the intestinal tract. More preferably, the pharmaceutical composition is a source of AP comprised within an enteric coat.

Enteric coatings arrest the release of the active compound from orally ingestible dosage forms. Depending upon the composition and/or thickness, the enteric coatings are resistant to stomach acid for required periods of time before they begin to disintegrate and permit slow release of AP (drug) in the lower stomach or upper part of the small intestines. Examples of some enteric coatings are disclosed in U.S. Pat. No. 5,225,202 (incorporated by reference). Examples of enteric coatings comprise beeswax and glyceryl monostearate; beeswax, shellac and cellulose, optionally with neutral copolymer of polymethacrylic acid esters; copolymers of methacrylic acid and methacrylic acid methylesters or neutral copolymer of polymethacrylic acid esters containing metallic stearates (for references enteric coatings see: U.S. Pat. Nos. 4,728,512, 4,794,001, 3,835,221, 2,809,918, 5,225,202, 5,026,560, 4,524,060, 5,536,507). Most enteric coating polymers begin to become soluble at pH 5.5 and above, with a maximum solubility rates at pH above 6.5. Enteric coatings may also comprise subcoating and outer coating steps, for instance for pharmaceutical compositions intended for specific delivery in the lower GI tract, i.e. in the colon (pH 6.4 to 7.0, ileum pH 6.6), as opposed to a pH in the upper intestines, in the duodenum of the small intestines the pH ranges 7.7-8 (after pancreatic juices and bile addition). The pH differences in the intestines may be exploited to target the enteric-coated AP composition to a specific area in the gut. It also allows the selection of a specific AP enzyme that is most active at a particular pH in the intestine. For instance calf intestinal AP (CIAP) and human placenta AP HPLAP) are most active at alkaline pH 8.2 in the small intestine, duodenum, jejunum and ileum, whereas milk-derived AP and Bone/Liver/Kidney or Tissue non-specific AP (TNS-AP) are most active at neutral pH and better suited for treatment of the colon (pH 7.4).

The most preferred mucosal tissues to be treated according to the current invention are the mucosal tissues lining the intestinal tract body cavities. Orally administered AP is delivered at the mucosal tissues of the GI tract, which comprises the esophagus, stomach, the small intestines or bowel, (duodenum, jejunum, ileum) and large intestines or colon (cecum, ascending colon, transverse colon, descending colon, sigmoid colon, rectum and anus). Within the scope of the current invention, also mucosal tissues lining the mouth, the ducts of the bile and the pancreas are part of the intestinal tract and may be treated according to methods of the current invention.

The compositions comprising a source of AP according to the current invention are particularly suited for oral administration to prevent treat, reduce, treat or alleviate inflammatory diseases of the gastrointestinal tract. Inflammatory diseases of the gastrointestinal tract may be induced and/or exacerbated significantly by the influx of LPS. A reduction in the amount of toxic LPS in the lumen of the intestines by administration of sources of AP will, through detoxification of the lipid A moiety of LPS, result in a corresponding decrease in the systemic influx of toxic LPS in the circulation of a subject. In a most preferred embodiment, the oral administration of sources of AP are particularly preferred for the prophylaxis or treatment of the following inflammatory disease of the gastrointestinal tract: Crohn's disease, colitis, (necrotizing) enterocolitis, colitis ulcerosa, hepatobiliary disease, hepatitis B, hepatitis C, liver cirrhosis, liver fibrosis, bile duct inflammation, biliary obstruction, pancreatitis, acute pancreatitis, peritonitis and periodontal disease.

In another embodiment of the invention, a source of AP is orally administered to subjects who suffer from an increased mucosal permeability of the gastrointestinal tract. Increased mucosal permeability of the GI tract is often the result of a decreased perfusion or ischemia of the intestines. Ischemia, a lack of oxygen supply by the bloodstream, may be caused by heart failure, injuries, trauma or surgery. Ischemia of the intestines results in a malfunctioning of the mucosa and a consequential increase in the influx or translocation of toxic LPS from the gut, resulting in both local and systemic toxicity and inflammation. The toxicity and inflammatory response may even further enhance the mucosal permeability, resulting in a vicious circle. Increased mucosal permeability of the GI tract may be the result of inflammatory bowel diseases or other pathological conditions of the GI tract. Oral administration of sources of AP according to the current invention will significantly reduce or abolish this increased influx of toxic LPS by detoxification of LPS in the lumen of the intestinal cavities. Exogenous administration of AP will break the vicious circle of LPS influx through the mucosa, inflammation and enhanced permeability of the mucosa resulting in an enhanced LPS influx. Decreased perfusion or ischemia of the intestines and a concomitant increased LPS influx is observed by the following group of diseases or conditions: burns, trauma and/or wounds which may result from accidents, gunshot or knife wounds, surgery, and in particular surgery with cardiopulmonary bypass. Also malfunctioning of the heart function, such as congenital heart disease, congestive heart failure, coronary heart disease and ischemic heart disease may result in ischemia of the intestines and an increased influx of LPS. It is one embodiment of the current invention to treat subjects suffering from this group of diseases and conditions with timely and regular oral administration of compositions comprising a source of AP to prevent or reduce LPS influx through the intestinal mucosa with an enhanced permeability for LPS.

In another embodiment the current invention is aimed at providing a source of AP at the mucosal lining the respiratory tract. The respiratory tract is another body cavity with a mucosal lining that is exposed to the toxic effects of LPS. LPS, either free or associated with inhaled bacteria, enters the respiratory tract bronchial and pulmonary mucosa via normal respiration, by inhalation of e.g., dust-particles, or from infections of the respiratory tract and mucosal tissues with gram negative bacteria. In addition, tobacco is known to be a rich source of LPS and smoking, either passive or active, may further contribute significantly to the LPS burden of the bronchial and pulmonary mucosa. Under normal conditions this LPS is detoxified by the local mucosal immune defense system in the respiratory tract. Therefore, in another preferred embodiment the current invention pertains to the administration of a source of AP via inhalation to the bronchial and pulmonary mucosa to prevent or reduce LPS influx through the mucosa of the respiratory tract for those conditions where the normal defense responses to LPS are malfunctioning. The current invention also provides compositions suitable for the delivery of AP at the bronchial and pulmonary mucosa. These compositions are preferably administered to for the prophylaxis or treatment of inflammatory diseases of the respiratory tract. In a most preferred embodiment pulmonary administration of a source of AP according to the current invention is applied to treat or prevent a disease selected from the group consisting of pneumonia, lung infections, asthma, CARA, cystic fibrosis, bronchitis and emphysema. The current invention also provides spraying devices, loaded with a composition comprising a source of AP and optionally various excipients such as propellants, carriers, nebulizers and/or diffusers, suitable for the administration of AP at the pulmonary and bronchial mucosa. Spraying devices, inhalators and nebulizers are known in the art of pharmaceutical formulation and will be obvious to the skilled artisan. See, Gennaro, AR, Remington: The Science and Practice of Pharmacy, Lippincott Williams & Wilkins Publishers; 21^{st} Ed, 2005 (or later edition)

In yet another embodiment, the current invention is aimed at the topical administration of a source of AP at a mucosal layer lining a body cavity. In a preferred embodiment the body cavity is the nasal cavity, oral cavity, vagina or rectum. Topical administration of a source of AP at a mucosal tissue lining a body cavity is preferably applied to treat local or systemic inflammatory diseases, and it is particularly preferred for the treatment or prophylaxis of infections of the nasal, vaginal, oral or rectal cavities, sexually transmitted diseases and infections, urinary tract infections, bladder infections and periodontal disease.

The current invention also provides compositions comprising a source of AP, amongst which are pharmaceutical and nutraceutical compositions comprising a source of AP. The compositions may optionally comprise pharmaceutically acceptable excipients, stabilizers, activators, carriers, permeators, propellants, disinfectants, diluents and preservatives. Suitable excipients are commonly known in the art of pharmaceutical formulation and may be readily found and applied by the skilled artisan, references, for instance, Gennaro, A R (supra). In a preferred embodiment the compositions comprising a source of AP are suitable for oral administration and comprise an enteric coating to protect the AP from the adverse effects of gastric juices and low pH. Enteric coating and controlled release formulations are well known in the art (references as described above). Enteric coating compositions in the art may comprise of a solution of a water-soluble enteric coating polymer mixed with the active ingredient(s) such as AP and other excipients, which are dispersed in an aqueous solution and which may subsequently be dried and/or pelleted. The enteric coating formed offers resistance to attack of AP by atmospheric moisture and oxygen during storage and by gastric fluids and low pH after ingestion, while being readily broken down under the alkaline conditions which exist in the lower intestinal tract.

AP containing compositions for the delivery of AP at mucosal tissues for detoxification of LPS according to the current invention preferably comprise an eukaryotic AP, more preferably a mammalian AP, which may be of the types tissue non-specific AP, such as liver-bone or kidney type, or tissue specific such as placental AP and intestinal AP. Most preferably the mammalian AP is a human or a bovine AP.

In a preferred embodiment of the current invention the source of AP is AP which is preferably produced or isolated from milk, preferably bovine milk. The milk may be obtained from animals that have been bred or genetically modified to produce elevated levels of AP in their milk as compared to wild-type animals. The preparation of AP enriched fractions from milk is known in the art. For instance the milkfat globule membrane enriched or derived fraction is the preferred AP enriched milk fraction and may be routinely obtained by conventional skimming of raw milk. AP isolated from milk may be formulated in pharmaceutical compositions and in food compositions or in nutraceuticals.

In a preferred embodiment the AP containing composition for oral administration of AP to the mucosa of the gastrointestinal tract according to the current invention is a food product or nutraceutical enriched for AP. In one embodiment the food product may be a plant, fruit or vegetable, optionally genetically modified to contain an enhanced level of AP. In another embodiment the AP containing food product or nutraceutical is a dairy product. In particular preparations and compositions containing non-pasteurized milk or fractions thereof, preferably bovine milk, contain high levels of AP and are particularly suited for oral administration as a source of AP according to the current invention.

The current invention also pertains to a method for the preparation of an AP enriched dairy product, preferably milk, a milk fraction or milk product. The method comprises the fractionation of raw milk, preferably bovine milk, pasteurization of the fractions not containing or not rich in AP and reformulating said fractions with the unpasteurized, AP rich fractions, to obtain a less perishable and AP enriched dairy product. The non pasteurized AP rich fractions may be sterilized by other means, such as, but not limited to, irradiation with UV-, X- or gamma-rays, filtration, pressure, osmotic pressure, chemicals or antibiotics, ensuring that the AP enzyme remains substantially active and that the milk fraction becomes substantially sterile. This dairy product may be used in compositions or administered directly to subjects suffering from or at risk of developing an LPS mediated or exacerbated disease and/or inflammation. However, the AP enriched dairy product may also be offered to healthy subjects as a pharmaceutical or nutraceutical product for the reduction of toxic LPS in the gastrointestinal tract and for the reduction of LPS influx through the gastrointestinal mucosa.

The invention further provides a composition comprising a source of alkaline phosphatase (AP) that is suitable for preventing or reducing lipopolysaccharide (LPS)-induced toxicity at a mucosal surface when the AP is delivered to the mucosa of a body cavity, which composition optionally further comprises a pharmaceutically acceptable:(i) stabilizer, (ii) activator, (iii) carrier, (iv) permeator, (v) propellant, (vi) disinfectant, (vii) protectant, (viii) diluent, (ix) nutrient or (x) another excipient, that promotes AP delivery to said mucosa. In a preferred embodiment, a composition according to the invention is provided, wherein the AP is a mammalian intestinal AP, a tissue non specific AP, a placental AP or a liver AP.

In another preferred embodiment, a composition according to the invention is provided, wherein the AP is of human or bovine origin.

In another embodiment, a composition according to the invention is provided, wherein the source of AP is a purified AP, an AP-enriched food product or an AP-enriched nutraceutical suitable for oral ingestion and delivery of the AP to the mucosal lining of the gastrointestinal (GI) tract. In a more preferred embodiment, the food product is a plant, a vegetable or a fruit that is optionally genetically modified to comprise and enhanced level of AP. In another more preferred embodiment, the food product is a dairy product, preferably a non-pasteurized or partially pasteurized milk or a milk fraction, preferably the milk fat globule membrane fraction.

In yet another embodiment, a composition according to the invention is provided, wherein the source of AP is enterically coated for oral administration and delivery to the GI mucosa.

In a particular embodiment, the invention provides an inhalation or spray device loaded with a composition according to the invention and a propellant and/or a nebulizer.

The invention also provides a method for preventing or reducing LPS toxicity at a mucosal surface of a mammalian body cavity in a subject, comprising administering to the subject in need thereof the composition according to the invention. In a preferred embodiment, the prevention or reduction of LPS toxicity is for prophylaxis or treatment of an LPS-mediated or LPS-exacerbated disease or condition.

In a preferred embodiment, a method according to invention is provided, wherein the LPS-mediated or LPS-exacerbated disease or condition is an inflammatory bowel disease, sepsis or septic shock, systemic inflammatory response syndrome, meningococcemia, trauma or hemorrhagic shock, a burn injury, cardiovascular surgery, cardiopulmonary bypass surgery, liver surgery, a liver transplant, liver disease, pancreatitis, necrotizing enterocolitis, periodontal disease, pneumonia, cystic fibrosis, asthma, coronary heart disease, congestive heart failure, renal disease, hemolytic uremic syndrome, a condition requiring kidney dialysis, an autoimmune disease, cancer, Alzheimer's disease, rheumatoid arthritis, or systemic lupus erythematosus.

In a preferred embodiment, the composition is administered orally. In another preferred embodiment, a method according to the invention is provided, wherein the mucosal surface is in the GI tract. In a more preferred embodiment, the composition is administered for the prophylaxis or treatment of a GI tract inflammatory disease. Even more preferred is a method according to the invention, wherein the GI tract inflammatory disease is selected from the group consisting of: inflammatory bowel disease, Crohn's disease, colitis, ulcerative colitis, hepatobiliary disease, hepatitis B, hepatitis C, liver cirrhosis, liver fibrosis, bile duct inflammation, biliary obstruction, pancreatitis, peritonitis, periodontal disease, and enterocolitis/necrotizing enterocolitis.

In another preferred embodiment a method according to the invention is provided, wherein the GI tract is more sensitive to LPS as a result of enhanced mucosal permeability of LPS due to (i) decreased intestinal perfusion or (ii) intestinal ischemia. Preferably, the decreased perfusion or ischemia is a result of cardiopulmonary bypass surgery, trauma or wounding, burns, cardiac surgery, congenital heart disease, congestive heart failure, coronary heart disease, or ischemic heart disease.

In yet another preferred embodiment, a method according to the invention is provided, wherein the composition is administered topically to said mucosa. Even more preferred, the composition is administered to nasal mucosa, oral mucosa, vagina mucosa, or rectal mucosa.

In an even more preferred embodiment, the composition is administered for treating a local or systemic inflammatory disease. In another even more preferred embodiment, a method according to the invention is provided, wherein the subject has a disease or disorder selected from the group consisting of a nasal infection, an oral infection, a vaginal infection or vaginitis, a rectal infection, a urinary tract infection, a sexually transmitted disease, and periodontal disease.

In another preferred embodiment, a method according to the invention is provided, wherein the composition is administered by inhalation. In a more preferred embodiment, the body cavity is respiratory tract mucosa. Even more preferred, the composition is administered for the prophylaxis or treatment of an inflammatory disease of the respiratory system.

In another preferred embodiment, a method according to the invention is provided, wherein the subject has a disease selected from the group consisting of pneumonia, a lung infection, asthma, cystic fibrosis, bronchitis, and emphysema.

The invention is further illustrated by the following non-limiting examples. The examples do not limit the scope of the invention in any way.

### Example 1

The current invention, and in particular the effectiveness of AP enzymes, preparations and compositions, and different modes of administration of AP may be tested in various animal models for inflammatory bowel diseases that are known in the art. Animal models mimicking human IBD comprise antigen-induced colitis and colitis induced by microbials; other inducible forms of colitis, colitis induced by chemicals (e.g., trinitrobenzene sulfonic acid (TNBS); Montfrans et al., 2002), immunological and physical and genetic colitis models (transgenic and knock-out models, see for instance SCID mice, Davis et al., 2003, IL-10 KO mice, Rennick et al., 2000, SAMP1/Yit mouse, Kosiewicz, et al., 2001 and Strober et al., 2001); adoptive transfer models and spontaneous colitis models (Kosiewicz et al., 2001).

The chemically induced Dextran Sulfate Sodium (DSS) colitis model was originally described by Okayasu et al., Gastroenterology, 199, 98:694-702, and is a model for human ulcerative colitis. The model comprises acute and chronic ulcerative colitis in mice caused by administration of 3-10% DSS in their drinking water. The morphological changes and changes in the intestinal microflora are similar to those seen in clinical cases of ulcerative colitis. The colon damage develops due to a toxic effect of DSS on the epithelial cells and to phagocytosis by lamina propria cells, resulting in production of TNF-α and IFN-gamma.

### Experimental Design for Acute DSS Colitis:

DSS (MW 40.000 obtained from ICN Chemicals) is dissolved in acidified drinking water in a concentration of 5% (w/v) and given ad libitum to female Balb/c mice (Harlan). The solution is refreshed every day. After 7 days of treatment, treated and control mice may be sacrificed and the intestines analyzed. The total colon is dissected (from cecum to rectum) and its length is recorded. About half of the colon is frozen in liquid nitrogen and cryo sections are made for morphology (HE staining). Also small parts of the spleen and the liver are snap-frozen in liquid nitrogen for immunohistochemical purposes. A small part of the colon is used to prepare tissue homogenates for cytokine measurements. Small colon strips are cultured in RPMI/10% FCS for 24 h in absence or presence of LPS. Cytokine secretion (TNFα; IL-1β; IFNγ) in the supernatant is measured using specific ELISA assays. Spleen and mesenteric lymph-nodes are dissected and squeezed to prepare single cell suspensions. 4 Peyer's Patches near the colon are dissected and single cell suspensions are made by use of collagenase. Cells are characterized using flow-cytometric techniques. Spleen cells are cultured for 24 h in RPMI/10% FCS in absence or presence of LPS or Con A. Cytokine secretion (TNF a; ILO; IFN gamma) in the supernatant is measured using specific ELISA assays. Feces are collected and cultured on McConkey agar plates for Enterobacteriaceae contents. For total aerobic bacteria content, feces are cultured on blood agar plates.

### Results

The assays described above are used to determine the effectiveness of compositions comprising AP in vivo. Reductions in cytokine secretion are observed; decreases in TNF α, I1-10 and IFNγ levels are measured in the inflamed intestines upon oral administration of the alkaline phosphatase rich milkfat globule membrane fraction of bovine milk.

### Example 2

### AP-Treated Mice Develop Less Severe Colitis after TNBS or DSS Treatment

### Materials and Methods:

### Experimental Design:

Three independent experiments were performed. For the first DSS experiment 42 eight-week old wild type C57BL/6 mice were obtained and for the TNBS experiment 20 eight-week old wild type BALB/c mice were obtained, from Charles River and from Harlan Nederland (Horst, The Netherlands), respectively. For a second DSS experiment, 72 eight week old C57BL/6 mice were obtained from Charles River Nederland. During the experiments, the mice were housed under standard conditions and they were allowed free access to water and food.

In the first experiment with C57BL/6 mice, colitis was induced by administration of 1.5% (n=18) or 2.5% (n=20) dextran sulfate sodium (DSS) in the drinking water of the mice for one week.

In the BALB/c mice, colitis was induced by rectal administration at day zero and seven of 1 mg 2,4,6-trinitrobenzene sulfonic acid (TNBS) (Sigma Chemical Co, St. Louis, Mo., USA) dissolved in 40% ethanol in PBS using a vinyl catheter that was positioned three centimeters from the anus. Preceding to the instillation, the mice were anaesthetized using isoflurane (1-chloro-2,2,2-trifluoroethyl-isofluranedifluoromethyl-ether) (Abbott Laboratories Ltd., Queen-borough, Kent, UK) and after the instillation the mice were kept vertically for 30 seconds. After 48 hours of the second TNBS administration, the mice were sacrificed.

During the induction of colitis, ten BALB/c and twenty C57BL/6 mice received orally 100 Units of alkaline phosphatase solved in 100 μl of 100 mM Tris (pH 7.8) once a day; the other mice received exclusively 100 μl of 100 mM Tris (pH 7.8). Four C57BL/6 mice were used as a reference control: they got no colitis and no treatment.

For a second DSS experiment, colitis was induced in C57BL/6 mice (n=48) by administration of 2% DSS in the drinking water for 5 days. 24 of these mice received 100 Units of alkaline phosphatase in 250 μl of 100 mM Tris (pH 7.8) once a day from day 5 up to day 14, whereas the other 24 mice received vehicle alone. A group of 24 mice that received normal drinking water and vehicle only served as reference control. This setup was used to investigate the use of AP as a rescue drug, once colitis is established.

In all experiments, the weight and temperature of the mice were recorded daily. After sacrificing the mice, caudal lymph node (CLN) and colon were obtained from the mice. Through a midline incision, the colons were removed and opened longitudinally. After removing the fecal material, the weight of the colons was measured and used as an indicator of disease-related intestinal thickening. The colons were divided in two parts, one of which was used for histological analysis and the other for cytokine detection.

### Histological Analysis:

The longitudinally divided colons were fixed in 4% formaldehyde embedded in paraffin for routine histology. Three transverse slices (5 μm), taken from each colonic sample, were stained with hematoxylin-eosin (H&E) and examined by light microscopy. Colonic inflammation was evaluated in a blind manner by estimating the 1) percentage of involved area, 2) the amount of follicles, 3) edema, 4) fibrosis, 5) erosion/ulceration, 6) crypt loss and 7) infiltration of granulocytes and 8) monocytes with a maximal score of 26.

The percentage of area involved and the crypt loss was scored on a scale ranging from 0 to 4 as follows: 0, normal; 1, less than 10%; 2, 10%; 3, 10 to 50%; 4, more than 50%. Follicle aggregates were counted and scored as follows: 0 point, 0-1 follicles; 1 point, 2-3 follicles; 2 point, 4-5 follicles; 3 point, more than 6 follicles. Erosions were defined as 0 if the epithelium was intact, 1 for ulceration that involved the lamina propria, 2 ulcerations involving the submucosa, and 3 when ulcerations were transmural. The severity of the other parameters was scored on a scale 0 to 3 as follows: 0, absent; 1, weak; 2, moderate; 3, severe.

### Cell Culture:

Caudal lymph node cells of TNBS mice were isolated by passing the lymph node through a 40 μm filter cell strainers (Becton/Dickson Labware, N.J., USA). The isolated lymphocytes were suspended in 4 ml RPMI 1640 medium, including L-glutamine, 10% fetal calf's serum (FCS) and antibiotics (Penicillin G sodium 10000 U/ml, Streptomycin sulfate 25 μg/ml. Amphotericin B 25 μg/ml; Gibco/BRL, Paisley, Scotland). The cells were counted and added to flat-bottom 96-well plates at 2×10⁵ cells per well in a total volume of 200 μl of the same medium. The cells were cultured in the presence of immobilized α-CD3 (1:30 concentration; 145.2 C11 clone) and soluble α-CD28 (1:1000 concentration; PharMingen) for 48 hours at 37° C. The supernatant was collected and used for a cytokine bead assay (CBA).

### Homogenization and Enzymatic Determination

Swiss roles of colonic samples that were taken 6 cm from the anus were frozen in the nitrogen. Homogenates were made with a tissue homogenizer in 9 volumes Greenberger lysis buffer (300 mmol/L NaCl, 15 mmol/L Tris, 2 mmol/L MgCl2, 2 mmol/L Triton X-100 (Sigma, St. Louis, Mo.), Pepstatin A, Leupeptin, Aprotinin (Roche, Mannheim, Germany), all 20 ng/mL; pH 7.4). The tissue was lysed for one hour on ice and centrifuged for 7 minutes at 3000 rpm and for 10 minutes at 14000 rpm. The supernatant was collected and stored frozen until the day of enzymatic determination and BD cytokine bead array analysis.

The feces of the mice were collected during the period of colitis. The weight of the feces was recorded and suspended in 0.6 ml of 50 mM glycine buffer with 0.5 mM MgCl2 (pH=9.6 at 25° C.). After centrifugation (10′, 13000 rpm), the supernatant was stored frozen until the day of determination of alkaline phosphatase activity.

The activity of alkaline phosphatase in the colon and feces was measured spectrophoto-metrically, using p-nitrophenyl phosphate as a substrate in 50 mM glycine buffer with 0.5 mM MgCl₂ (pH=9.6 at 25° C.). Enzymatic activity was expressed in mU/ml for the colon homogenates and in mU/mg for the feces.

### Cytokine Bead Assay (CBA)

A cytokine bead assay was performed to determine simultaneously the production of TNF-α, IFN-γ, IL-2, IL-4 and IL-5 in colon homogenates and CLN cell culture supernatant according to the manufacturers recommendations of Becton Dickinson (BD) and as de-scribed elsewhere (37, see 2.2). Briefly, particles (polystyrene beads) were dyed to five fluorescence intensities. The proprietary dye had an emission wavelength of ˜650 nm (FL-3). Each particle was coupled via a covalent linkage based on thiol-maleimide chemistry with an antibody against one of the five cytokines and represented a discrete population, unique in their FL-3 intensity. The Ab-particles served as a capture for a given cytokine in the immuno-assay panel and could be detected simultaneously in a mixture. The captured cytokines were detected via direct immunoassay using five different antibodies coupled to phycoerythrin (PE), which emitted at ˜585 nm (FL-2). The standards ranging from 0 to 2000 pg/ml were mixtures of all five cytokines, so that five standard curves were obtained. For each sample and cytokine standard mixture, 10 μl of capture Ab-bead reagent, samples or standard and detector Ab-PE reagent were incubated for three hours and were washed to remove unbound detector Ab-PE reagent before data acquisition using flow cytometry. Two-color flow cytometric analysis was performed using a FACScan® flow cytometer (Becton Dickinson Immunocytometry Systems (BDIS), San Jose, Calif.). Data were acquired and analyzed using Becton Dickinson Cytometric Bead Array (CBA) software.

### Statistical Analysis

All data were expressed as the mean±standard deviation. Where indicated Student's t test was used to calculate statistical significance for difference in a particular measurement between different groups. Values of p<0.05 were considered statistically significant (*p<0.05).

### Results

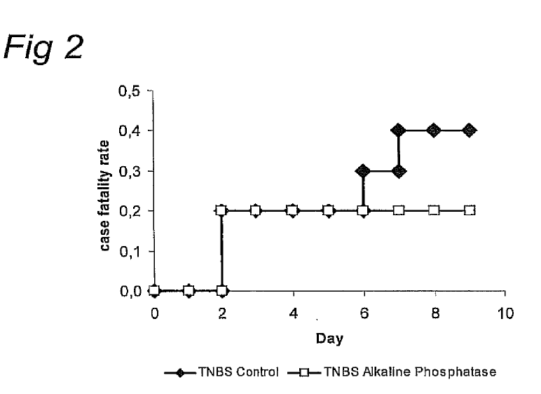

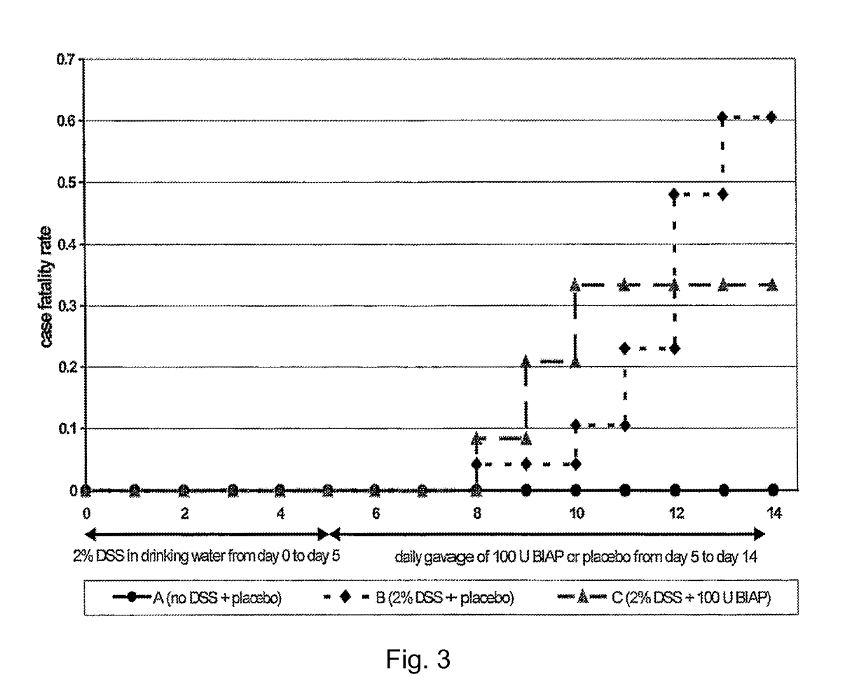

To investigate whether oral AP has therapeutic potential in IBD, the TNBS- and DSS-induced colitis in mice were used as models. As predicted, intrarectal instillation of TNBS and oral administration of DSS resulted in diarrhea and wasting disease. At day two, two of the ten mice in each of the two treatment groups died (see FIGS. 2 and 3) indicating that AP does not prevent mortality due to the early inflammatory response Two or three days after the initial intrarectal administration of TNBS a delayed hypersensitive responsive type 4 reaction is activated and causes most of the inflammation, which leads to additional mortality. Although AP treatment could not prevent the death of the mice during this first reaction, it prevented additional deaths due to the secondary inflammatory response.

In the groups that received 2% DSS in their drinking water for 5 days with subsequent AP gavage for up to 14 days, 33% of the mice died between day 8 and 10 in the AP treated group. Thereafter no mice died in this group. In the placebo treated group, however, in total 60% of the mice died from day 8 onwards until day 14. Similar to the TNBS model, AP is able to reduce mortality in the later stage of DSS-colitis, but not in the induction phase.

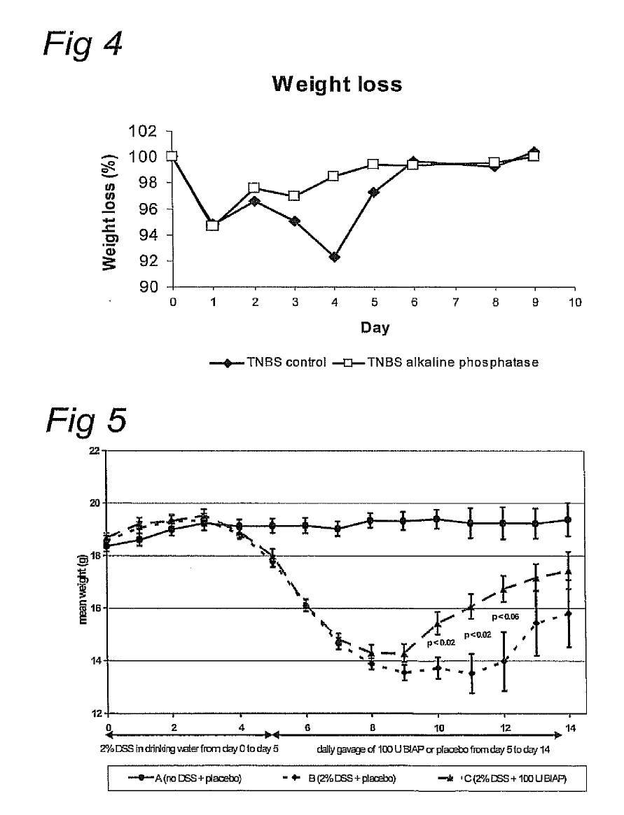

After the first administration of TNBS the weight of the mice has decreased at the first day in both the AP-treated as in the control mice (see FIG. 4). The weight of the control mice, however, was decreased to 92% of their initial weight at day four, whereas the weight of the AP-treated mice was increased to above 98% of their initial weight and was stabilized. At the sixth day, the control mice also reached their initial weight. The effects on weight are thus almost synchronized to the described survival benefits.

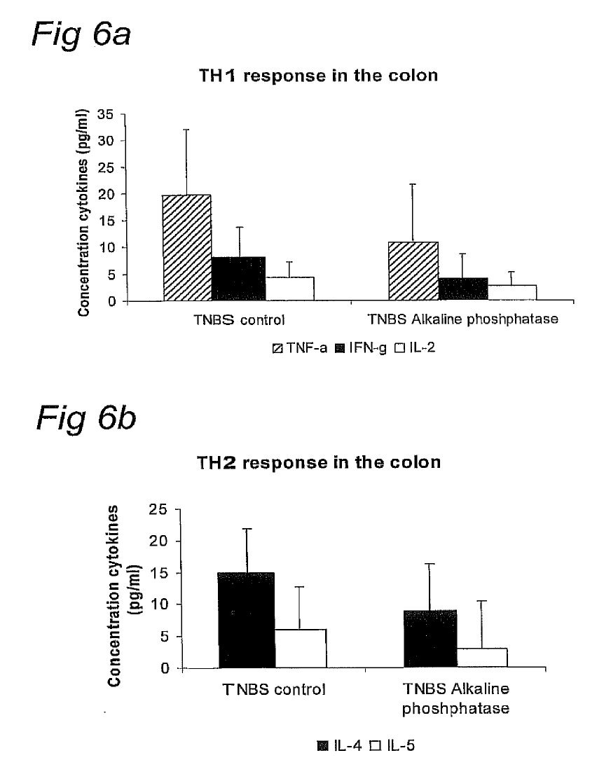

In the DSS studies, only the second group that received 2% DSS and was followed up to 14 days showed statistical significant differences on days 10, 11 and 12 (FIG. 5). The other two groups were followed only up to day 7 and showed, as the former group, no difference between AP treated and untreated groups until that day (data not shown).

### Example 3

### Cytokine Production and AP Activity were Decreased in Colons of AP-Treated Mice

Colon homogenates of TNBS and DSS mice were analyzed for the production of cytokines by a cytokine bead assay to investigate the size of the Th1 response. In contrast to the increased cytokine production in the CLN of AP-treated mice, the production of TNF-α, IFN-γ, IL-2, IL-4 and IL-5 was decreased in the colon homogenates of these mice compared to the control mice, however not significantly (see Table 2). The mice with 2.5% DSS-induced colitis confirm similar results, although these results did not reach statistical significance, too (not shown).

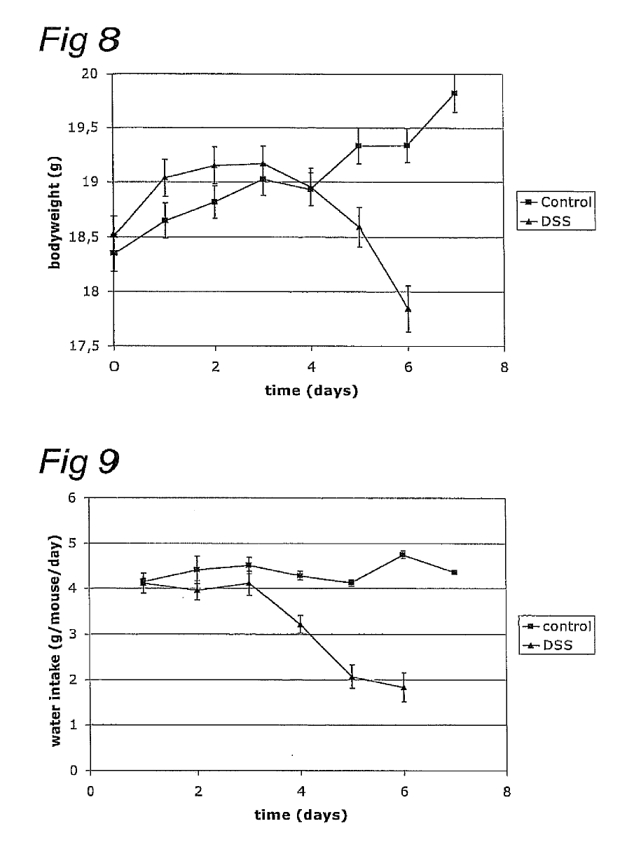

The production of IL-2, -4 and -5 in the 1.5% DSS mice were almost undetectable (data not shown). The production of TNF-α was decreased in the AP-treated mice compared to the DSS control mice, however not significantly (see FIG. 7). In case of the IFN-γ production, the differences were significant (p<0.05): the IFN-γ production was decreased from 100.5±82.3 pg/ml in DSS control mice to 31.6±21.3 pg/ml in AP-treated mice.

TABLE 2
Cytokine concentrations in colon homogenates measured by CBA

| | Control | AP-treated |
|---|---|---|
| Cytokine | TNBS mice | TNBS mice |
| TNF-α | 19.7 ± 12.4 | 10.8 ± 10.8 |
| IFN-γ | 8.2 ± 5.5 | 4.0 ± 4.6 |
| IL-2 | 4.4 ± 2.9 | 2.7 ± 2.6 |
| IL-4 | 15.0 ± 7.0 | 8.9 ± 7.5 |
| IL-5 | 5.9 ± 4.0 | 2.9 ± 3.8 |
| | Control | AP-treated |
| | 1.5% DSS mice | 1.5% DSS mice |
| TNF-α | 15.6 ± 5.1 | 13.1 ± 5.4 |
| IFN-γ | 100.5 ± 82.3 | 28.4 ± 19.8 |
| | Control | AP-treated |
| | 2.5% DSS mice | 2.5% DSS mice |
| TNF-α | 89.3 ± 122.3 | 60.7 ± 40.8 |
| IFN-γ | 130.0 ± 85.5 | 83.2 ± 90.0 |
| IL-2 | 13.2 ± 25.8 | 7.2 ± 6.0 |
| IL-4 | 23.4 ± 42.5 | 24.7 ± 19.2 |
| IL-5 | 19.5 ± 36.8 | 13.9 ± 12.5 |

### Example 4

### Single Oral Dose Pharmacokinetic Assay of BIAP in Mice

### Materials and Methods

Subject of investigation was the local and systemic bioavailability after single high dose application of oral BIAP (Bovine intestinal alkaline phosphatase). The test material was delivered as a solution and was stored at 4° C. until use. Dosing dilutions in autoclaved drinking water were prepared freshly on the day of treatment. Autoclaved drinking water was used as control solution.

The C57BL/6 mouse is a suitable rodent species for DSS-induced colitis and is acceptable to regulatory authorities for safety testing. The oral route of administration corresponds to the intended therapeutic use in humans.

The study room and cages were cleaned and disinfected. During the study, the room and cages were cleaned at regular intervals. The room temperature was adjusted to 22±3° C. and the relative humidity was kept between 30% and 70%. These parameters were monitored daily. Artificial light was set to give a cycle of 12 hours light and 12 hours dark with light on at 6:00 a.m. Air was changed about 8 times per hour in the animal room and filtered adequately. The animals were fed ad libitum with SDS D3 pellets, analyzed by the supplier for nutrients and contaminants. Drinking water sterilized by autoclaving was continuously available ad libitum via drinking bottles. Consumption is controlled visually on a daily basis.

TABLE 3
Experimental groups and DSS pretreatment
were assigned as follows:

| | | | | Concentration | Volume |
|---|---|---|---|---|---|
| | | No. of | Card | (drinking | [ml/kg |
| Group | Treatment | Animals | labelling | water) | body wt] |
| Red | DSS | 29 | A-E (red) | 2% | See |
| | | | | | Table 4 |
| Green | — | 29 | A-E (green) | — | See |
| | | | | | Table 4 |

TABLE 4
Experimental groups and BIAP treatment were assigned as follows::

| | | | | Conc in | | |
|---|---|---|---|---|---|---|
| | From | | No. of | drinking | Volume [ml/kg | Time of sacrifice |
| Group | Cage | Treatment | animals | water | body wt | (min) |
| Control A | A green | Placebo | 5 | — | 12.5 | 150 |
| Control B | B green | BIAP | 3 | 6000 U/ml | 12.5 | 10 |
| Control C | B green | BIAP | 3 | 6000 U/ml | 12.5 | 30 |
| Control D | C green | BIAP | 3 | 6000 U/ml | 12.5 | 60 |
| Control E | C green | BIAP | 3 | 6000 U/ml | 12.5 | 90 |
| Control F | D green | BIAP | 3 | 6000 U/ml | 12.5 | 120 |
| Control G | D green | BIAP | 3 | 6000 U/ml | 12.5 | 180 |
| Control H | E green | BIAP | 3 | 6000 U/ml | 12.5 | 240 |
| Control I | E green | BIAP | 3 | 6000 U/ml | 12.5 | 360 |
| DSS A | A red | Placebo | 5 | — | 12.5 | 150 |
| DSS B | B red | BIAP | 3 | 6000 U/ml | 12.5 | 10 |
| DSS C | B red | BIAP | 3 | 6000 U/ml | 12.5 | 30 |
| DSS D | C red | BIAP | 3 | 6000 U/ml | 12.5 | 60 |
| DSS E | C red | BIAP | 3 | 6000 U/ml | 12.5 | 90 |
| DSS F | D red | BIAP | 3 | 6000 U/ml | 12.5 | 120 |
| DSS G | D red | BIAP | 3 | 6000 U/ml | 12.5 | 180 |
| DSS H | E red | BIAP | 3 | 6000 U/ml | 12.5 | 240 |
| DSS I | E red | BIAP | 3 | 6000 U/ml | 12.5 | 360 |

According to Table 4 all mice except those in the placebo groups in both control and DSS treated groups received a single oral dose of 75,000 U/kg BIAP per oral gavage (in 250 μl autoclaved drinking water). Placebo groups (n=5) received 250 μl autoclaved drinking water only. All animals were treated once. Before treatment with BIAP, some of the animals were treated with DSS (see Table 3).

After blood collection, intestines from the animal were prepared and feces collected from each one third part of the small intestine and from the colon separately. Each sample was dissolved by vigorous vortexing in 1 ml glycine buffer (25 mM; pH=9.6)

These samples were analyzed for alkaline phosphatase content using the an assay for the determination of alkaline phosphatase activity. Paranitrophenyl phosphate, which is colorless, is hydrolyzed by alkaline phosphatase at pH 9.6 and 25° C. to form free paranitrophenol, which is colored yellow. The reaction can be followed spectrophotometrically. The change in optical density at 405 nm per unit time is a measure of the alkaline phosphatase activity. The amount of enzyme causing the hydrolysis of one micromole of paranitrophenyl phosphate per minute at pH 9.6 and 25° C. is defined as one unit. The amount of units present in a sample can be calculated or calibrated against a curve of AP samples with a known AP concentration.

### Results

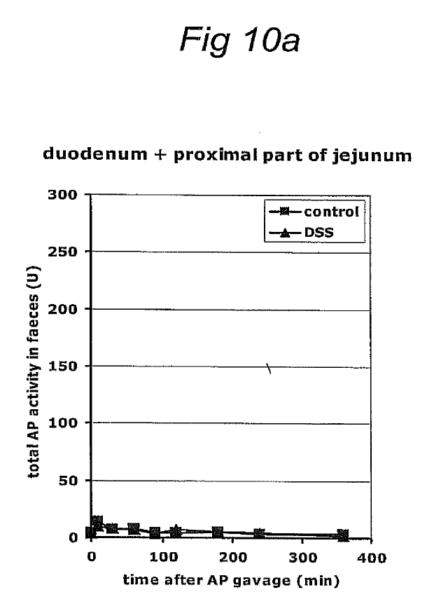

During the pretreatment phase with DSS, mice were weighed daily as an indication for development of colitis. Weight loss of about 10% compared with control mice is used to identify DSS induced colitis. On day 6, mice that were treated for 5 days with DSS had lost around 10% bodyweight (FIG. 8) and were used in the pharmacokinetic experiment to identify local and systemic bioavailability of administered BIAP during colitis. One day later, control mice were used in the same manner.

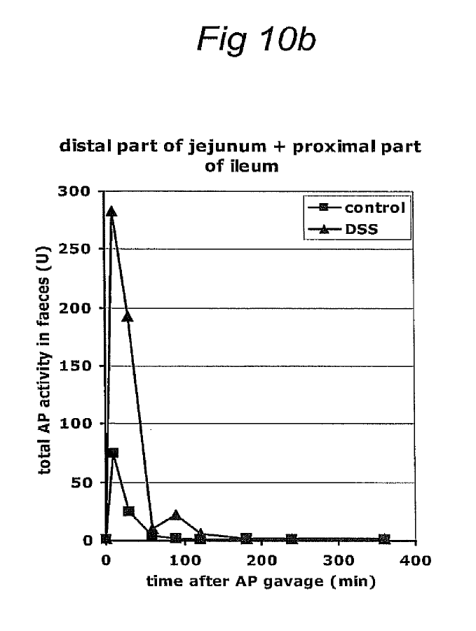

In order to analyze DSS uptake, drinking bottles were weighed daily before and after refilling of the bottles. The first three days, DSS treated animals consumed approx. the same amount of water as control animals did. Thereafter, DSS mice drank less, probably as a result of decreased well being. However, daily intake of DSS was about 40 mg/mouse, which was shown to be sufficient for induction of colitis in other experiments (see FIG. 9).

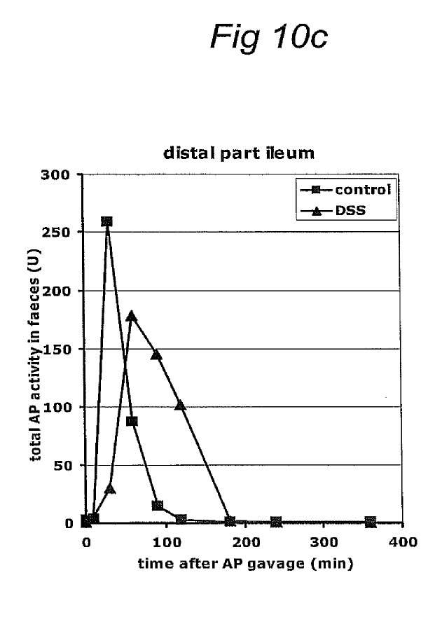

The objective of this study was to analyze the local bioavailability in the intestinal tract. For this purpose, on different time points mice were sacrificed and the intestinal tract from duodenum to colon isolated. The small intestine (inc. Duodenum) was divided in two three equal parts. Feces from each part and from the colon was extracted and alkaline phosphatase activity measured. In FIG. 10, the alkaline phosphatase activity in the different parts of the intestine at different time points is shown. No alkaline phosphatase could be measured at any time point in the duodenum and proximal part of the jejunum. This was probably due to the fact that passage through this part takes place within 10 minutes, the first sampling point. At 10 minutes both DSS as well as control animals have peak alkaline phosphatase levels in the distal part of jejunum and proximal part of ileum. Between half an hour and one hour, peak values of AP occur in the distal part of the ileum. In the colon, alkaline phosphatase is increased one and a half hour after gavage and after six hours, most alkaline phosphatase was either excreted or broken down.

Considering that a total of 1350 U was administered per mouse, local bioavailability is estimated in the distal part of the ileum and the colon at 200/1350=15% and 80/1350=6%, respectively. However these values are probably underestimation as they are based on mean peak values of 3 individual mice each (see discussion).

The objective of this study was to estimate the local bioavailability of BIAP after high oral dose administration. The local bioavailability was estimated at 15% in the distal part of the ileum and 6% in the colon. These values are probably grossly underestimated, because values are from individual mice at a given time point and do not represent alkaline phosphatase activity throughout the tract of one mouse followed in time. As a consequence, peak values may be missed in individual mice in a given part of the intestine at a given time point. However, the results as depicted in FIG. 10 clearly demonstrate local bioavailability of AP upon oral administration. These results underscore the feasibility of the method of the current invention to administer to a subject a source of alkaline phosphatase in order to prevent or reduce (toxic) LPS influx through a mucosal lining of a mammalian body cavity.

### Example 5

### Protective role of Alkaline Phosphatase in Necrotizing Enterocolitis

### Materials and Methods

### Animal Model

All animal protocols were approved by the institution Animal Care and Use Committee and followed a well-described neonatal rat model of NEC (Dvorak et al., Am J Physiol Gastrointest Liver Physiol 2002; Barlow B et al., J Pediatr Surg 1974; Barlow B et al., Surgery 1975; Nadler et al., J Surg Res 2000; Whitehouse I S et al., J Surg Res 2009). Litters of full-term Sprague Dawley rat pups (Harlan Laboratories, Madison, Wis.) were split into 5 experimental groups several hours after birth. Control pups remained with the mother, were breast fed, and kept at normoxia and normothermia. To induce NEC, the remaining four groups of experimental pups were housed separately from the mother in an Air-Shields T-100 infant transport incubator (Soma Technologies, Bloomfield, Conn.) set at 37° C. They were exposed to hypoxia by placing them in a room temperature Praxair Biosperix (Lacona, N.Y.) chamber, and subjected to 5% oxygen for 10 minutes, 3 times daily. Additionally, they were gavage-fed transorally with 0.2 ml formula 3 times per day using a 24-gauge blunt-tipped angiocatheter (Instech Solomon, Plymouth Meeting, Pa.). Neonatal rats were fed a base formula of Similac® infant formula (4 g, Abbott Laboratories, Columbus, Ohio) in Esbilac® canine milk replacement (20 ml, PetAg Inc., Hampshire, Ill.). The formula was supplemented with LPS (1 mg in 1.0 ml PBS, Escherichia coli, Sigma-Aldrich, St. Louis, Mo.), resulting in administration of 2 mg/kg LPS per feed. The first NEC group (NEC-1) received LPS with all three feeds. NEC-2 pups received LPS only during the noon and evening feeds. The base formula without LPS was administered during the morning feed in the NEC-2 group. The final two experimental groups were fed 4 glycine units of bovine calf iAP (Sigma-Aldrich, St. Louis, Mo.) in their morning feed. The iAP was either mixed with LPS formula (IAP-1) or base formula alone (iAP-2). These experimental groups were designed so that the NEC-1 and iAP-1 pups received a total of 3 doses of LPS per day, while NEC-2 and iAP-2 pups only received 2 doses of LPS. We chose this method to exclude the possibility that iAP dephosphorylates the LPS ex vivo prior to oral administration, thus essentially protecting the iAP-1 pups from receiving a functional 3^{rd} dose of LPS.

The pups and adult rats were sacrificed on day 4 after birth using intraperitoneal injections of a ketamine and xylazine mixture (100 mg/kg, 10 mg/kg) and lethal carbon dioxide inhalation, respectively. Laparotomies were performed and the entire intestinal tract from the distal esophagus to distal rectum was removed. First, short segments of duodenum and terminal ileum were harvested from the adult rats. Enteric contents were gently evacuated from the lumen and the intestinal tissue was placed in fresh Hanks balanced salt solution (HBSS, Sigma-Aldrich, St. Louis, Mo.) on ice. Second, a short segment of terminal ileum from the pups was harvested for histology grading of NEC. Tissues were fixed in zinc formalin (Richard-Allan Scientific, Kalamazoo, Mich.), processed and subsequently embedded in paraffin. Hematoxylin and eosin staining was performed on 4 micron thick sections. Tissues were graded using a 4-point scale previously described.(Dvorak et al., Am J Physiol Gastrointest Liver Physiol 2002; Nadler et al., J Surg Res 2000; Whitehouse I S et al., J Surg Res 2009). Second, the entire intestinal mesentery from the duodenum to rectum was carefully dissected from the bowel and discarded. The remaining intestinal tissue was placed in fresh Hanks balanced salt solution (HBSS, Sigma-Aldrich, St. Louis, Mo.) on ice and subsequently used for AP activity analysis as well as for Western blot analysis to detect AP expression.

### Protein Quantification

Intestines from adult rats and neonatal rat pups were placed in fresh HBSS (500 μl) in a 2 ml microfuge tube (Axygen Scientific, Union City, Calif.). The tissue was cut into small pieces using alcohol-treated fine scissors and homogenized while on ice (3 bursts, 10 seconds each burst) using a hand-held Polytron PT 1200 E homogenizer (Kinematica, Inc., Bohemia, N.Y.). Homogenates were lysed with MOPS buffer (Xu H et al., J Biol Chem 2007) (500 μl) containing protease and phosphatase inhibitors (Sigma-Aldrich, St. Louis, Mo.). The tubes were centrifuged at 14,000 rpm for 10 min at 4° C. to pellet cell debris. The supernatant was removed and saved for protein quantification, AP activity assay, and Western blot analysis. Protein content was quantified using the bicinchoninic acid (BCA) reagent per the manufacturer's protocol (Pierce Chemical Company, Rockford, Ill.).

### Alkaline Phosphatase Activity Assay

AP activity was measured using a colorimetric assay kit (BioVision, Mountain View, Calif.). Equal amounts of protein (0.5 μg) from the lysate of each intestinal tissue sample were analyzed and AP activity was calculated (U/ml) according to the manufacturer's protocol.

### Immunoflourescent Staining

Due to the lack of commercially available iAP antibodies that react with rat, antibodies directed to TNAP were used when analyzing intestinal tissue. Unstained 4 micron sections of terminal ileum from each neonatal specimen were used to detect AP immunoflourescently. The slides embedded in paraffin were soaked in 100% xylene for a total of 20 minutes and then underwent sequential washings with 100%, 95%, 75%, and 50% ethanol to achieve rehydration. Slides were blocked with 3% BSA (Sigma-Aldrich, St. Louis, Mo.) at room temperature for 20 minutes and then incubated with polyclonal rabbit AP antibody (1:50, Santa Cruz Biotech-nologies, Santa Cruz, Calif.) for 1 hour at 37° C. The slides were washed with PBS and then incubated with goat anti-rabbit Alexa Fluor 633 antibody (1:1000, Invitrogen, Carlsbad, Calif.) for 30 minutes at 37° C. Slides were mounted with a fluorescent mounting media (Dako North America, Carpinteria, Calif.) and imaged with a fluorescent microscope at 20× magnification.

### Western Blot Analysis

Equal volumes of 5× Laemmli sample buffer (Bio-Rad, Hercules, Calif.) were added to each protein sample. Purified bovine iAP was used as a positive control (BBI Enzymes, Inc., Madison, Wis.). MOPS lysis buffer was used to bring the total volume to 100 μl. Samples were heated at 95° C. for 5 minutes, cooled to room temperature (RT) and then loaded onto a 10% Tris-HCl-Criterion gel system (Bio-Rad, Hercules, Calif.). Tris-Glycine SDS running buffer was used as well as All Blue and Dual Rainbow markers (Bio-Rad, Hercules, Calif.). Protein was transferred to a 0.45 micron nitrocellulose membrane using the Criterion Blotting system (4° C., 55V for 2 hours, BioRad, Hercules, Calif.). The membrane was incubated for 1 hour at room temperature in TBS-T blocking buffer containing 5% non-fat dry milk (SantaCruz Biotechnologies, Santa Cruz, Calif.). Primary antibodies were a rabbit, polyclonal anti-AP antibody (1:1000, Santa Cruz Biotechnologies, Santa Cruz, Calif.) and mouse, monoclonal anti-β-actin antibody to control for loading (1:1000, Santa Cruz Biotechnologies, Santa Cruz, Calif.). Incubations were in PBS-T containing 5% BSA (Sigma-Aldrich, St. Louis, Mo.). The membranes were incubated overnight at 4° C. on an orbital shaker. Membranes were then washed with TBS-T and then incubated with the appropriate secondary antibodies [goat anti-rabbit IgG HRP and goat anti-mouse IgG HRP (1:5000 from Santa Cruz Biotechnologies, Santa Cruz, Calif.)] for 1 hour at RT. The membranes underwent serial washings with TBS-T. Bands were detected using ECL Plus™ and Hyperfilm ECL™ according to the manufacturer's instructions (Amersham/GE Healthcare, Piscataway, N.J.).

### Statistical Analysis

Computerized densitometry of western blot film using UN-SCAN-IT software (version 6.1, Silk Scientific, Inc., Orem, Utah) was used to quantify differences in band intensities. Values for AP bands were normalized using p-actin bands. Data are expressed as mean±SEM, Statistical analysis was performed using the Student's t-test with a p-value of <0.05 considered significant.

### Results

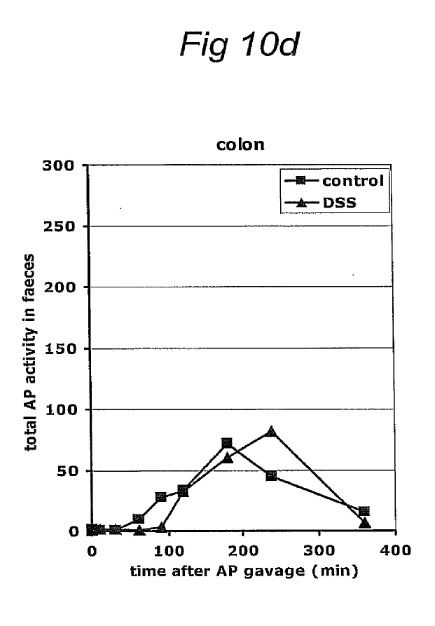

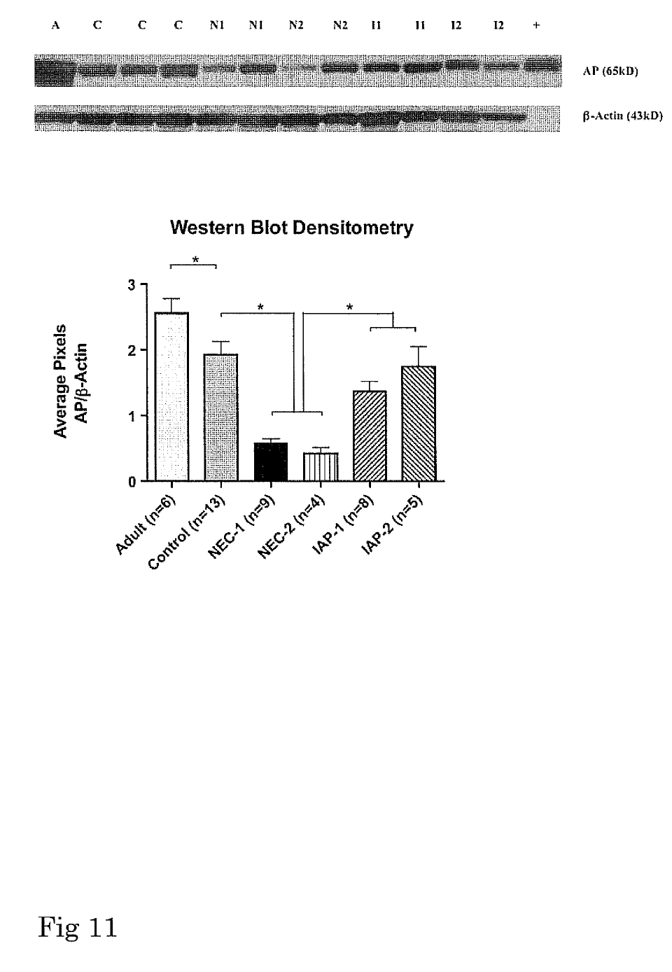

One of our hypotheses was that differences in iAP expression or function in newborns predispose them to increased risk of NEC in the neonatal period. To investigate this, we first compared AP expression and AP activity in healthy term newborn pups to the adult mothers (FIGS. 11 and 12, respectively). Due to the sheer bulk of the adult intestinal tract, only sections from the duodenum and terminal ileum were analyzed. Although AP protein expression did not differ between the adult duodenum and ileum, the AP activity was highest in the duodenum (0.315 vs. 0.072, not shown). Therefore, all comparisons to adult tissue in our results refer to the duodenum. FIG. 11 shows a representative western blot of AP and β-actin expression from a single experiment. Although there was variation in AP expression in the individual experiments, taken in aggregate there were significant differences in band densities amongst the experimental groups. The cumulative densitometry graph from all of our experiments is also shown in FIG. 11. AP protein expression was significantly increased in the mother's duodenum compared to expression in the pups intestine (p=0.05). Interestingly, the control pups had increased AP activity, although this did not reach statistical significance (0.315 vs. 0.668 U/ml, p=0.08) (FIG. 12). However, in both NEC-1 and NEC-2 pups AP protein (0.58 and 0.42 vs. 1.93 avg. pixels, p<0.001) and AP activity (0.253 and 0.308 vs. 0.668 U/ml, p<0.05) were significantly decreased in comparison to controls.

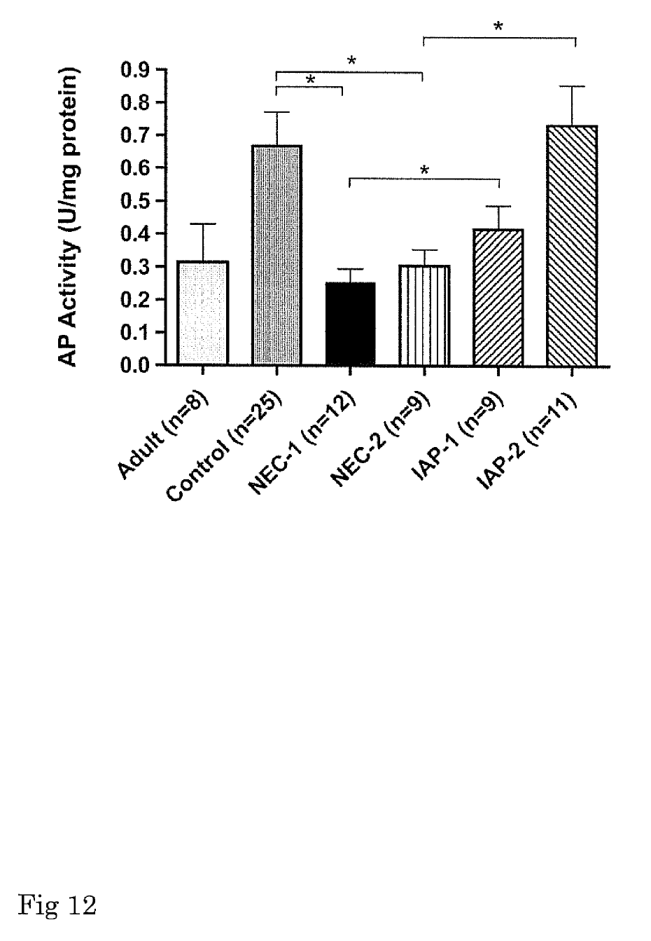

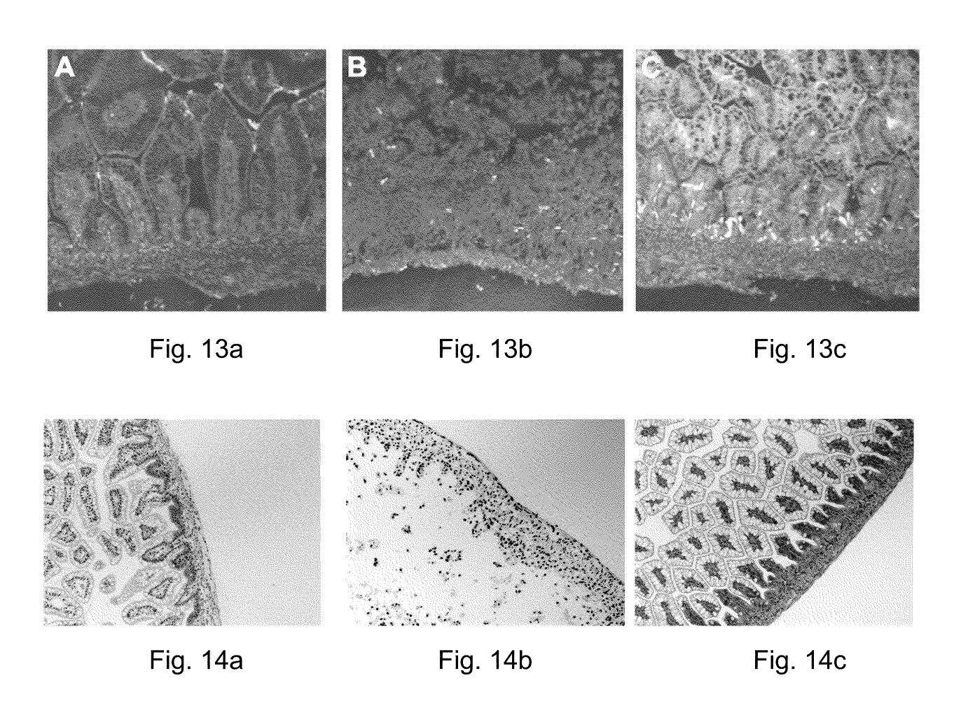

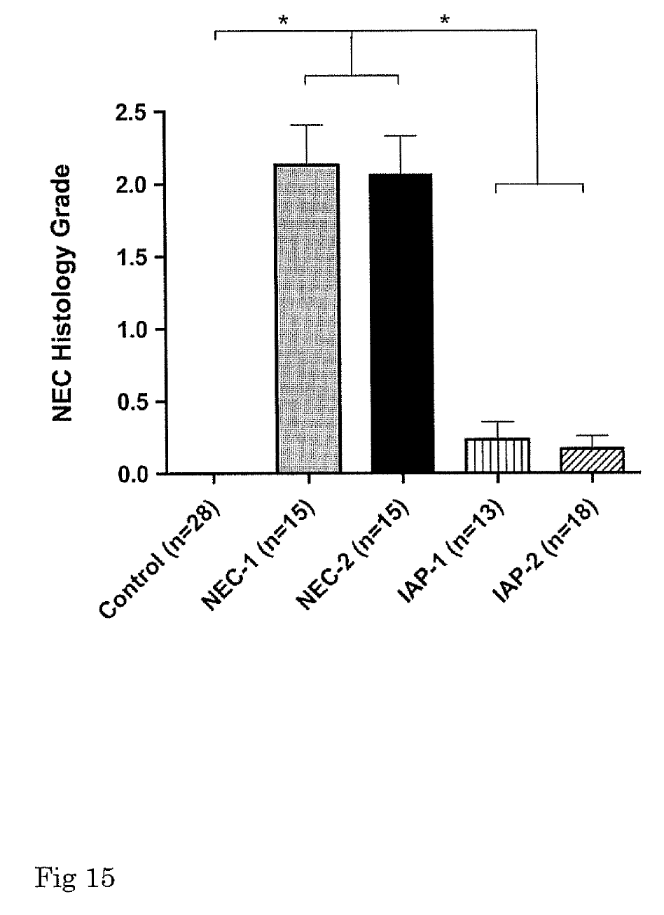

Next, we hypothesized that supplemental iAP would prevent development of NEC. We wanted to demonstrate that the iAP we administered in the feeds was not inactivated and degraded in the foregut. Therefore we performed immunofluorescence staining of the terminal ileum for AP in all of our pups. As shown in FIG. 13, the control pups had a bright signaling and the NEC pups had a very low signal. The NEC pups treated with iAP, however, had a very bright signal similar to the control pups. Similarly, when we compared the AP activity in the intestine we found that regardless of whether or not the iAP was administered with LPS, AP activity was significantly increased in iAP-1 and iAP-2 pups in comparison to NEC-1 and NEC-2 pups, respectively (0.253 vs. 0.415 and 0.308 vs. 0.783 U/ml, p<0.05) (FIG. 12). Lastly, we wanted to show that exogenous iAP prevents the development of NEC. As shown in FIGS. 14 and 15, control pups showed normal histology and an intestinal injury grade of 0 on a 4-point scale (Nadler E P et al., J Surg Res 2000). FIG. 14(b) shows a pup with grade 4 intestinal injury, which represents tissue observed in both NEC-1 and NEC-2 pups. Overall, NEC-1 pups displayed an average grade of 2.13 (range 1-4), which did not differ from NEC-2 pups (2.07, range 1-4, p=0.86) (FIG. 15). Pups treated with exogenous enteral iAP showed histology comparable to control pups (FIG. 14) and had low injury grades (iAP-1=0.231 and iAP-2=0.167, p=0.67) (FIG. 15). The average grade of injury in NEC-1 and NEC-2 pups was significantly increased when compared to both controls and iAP pups (all p-values<0.001).

### REFERENCES CITED

Alvaro D, Benedetti A, Marucci L, et al., The function of alkaline phosphatase in the liver: regulation of intrahepatic biliary epithelium secretory activities in the rat. Hepatology (United States), August 2000, 32 p174-84
Anh D J, Eden A, Farley J R. Quantitation of soluble and skeletal alkaline phosphatase, and insoluble alkaline phosphatase anchor-hydrolase activities in human serum. Clin Chim Acta. Sep. 25; 311: 137-48.
Barlow B, Santulli T V. Importance of multiple episodes of hypoxia or cold stress on the development of enterocolitis in an animal model. Surgery 1975; 77:687.
Barlow B, Santulli T V, Heird W C, Pitt J, Blanc W A, Schullinger J N. An experimental study of acute neonatal enterocolitis—the importance of breast milk. JPediatrSurg1974; 9:587.
Bentala H, Verweij W R, Huizinga-Van der Vlag A, van Loenen-Weemaes A M, Meijer D K, Poelstra K. Removal of phosphate from lipid A as a strategy to detoxify lipopolysaccharide. Shock. 2002 December; 18: 561-6.
Bentala, H., Verweij, W. R., Brands, R, Huizinga-VanderVlag A, Meijer, D K F, Poelstra, K. Intravenous administration of alkaline Phosphatase to mice challenged with E. coli lipopolysaccharide has prominent protective effects. Submitted for publication
Beumer C, Wulferink M, Raaben W, Fiechter D, Brands R, Seinen W. Calf intestinal alkaline phosphatase, a novel therapeutic drug for lipopolysaccharide (LPS)-mediated diseases, attenuates LPS toxicity in mice and piglets. J Pharmacol Exp Ther. 2003 November; 307(2): 737-44.
Bin-Nun A, Bromiker R, Wilschanski M, Kaplan M, Rudensky B, Caplan M, Hammerman C. Oral probiotics prevent necrotizing enterocolitis in very low birth weight neonates. J Pediatr 2005; 147:192.
Claud E C, Savidge T, Walker W A. Modulation of human intestinal epithelial cell IL-8 secretion by human milk factors. Pediatr Res 2003; 53:419.
Cnota J, Shetty A K, Land M H, Rouster-Stevens K, Woods C, Cannon ML. Sepsis associated with probiotic therapy lactobacillus. Pediatrics 2005; 15:178.
Paul H. Davis and Samuel L. Stanley, Jr., Breaking the species barrier: use of SCID mouse-human chimeras for the study of human infectious diseases, Cellular Microbiology (2003) 5:849-860.
Deng J T, Hoylaerts M F, De Broe M E, van Hoof V O. Hydrolysis of membrane-bound liver alkaline phosphatase by GPI-PLD requires bile salts. Am J. Physiol. 1996 October; 271(4 Pt 1): G655-63.
Dvorak B, Halpern M D, Holubec H, Williams C S, Me William D L, Dominguez J A, Stepankova R, Payne C M, McCuskey R S. Epidermal growth factor reduces the development of necrotizing enterocolitis in a neonatal rat model. Am J Physiol Gastrointest Liver Physiol 2002; 282:156.
Eichbaum E B, Harris H W, Kane J P, Rapp J H. Chylomicrons can inhibit endotoxin activity in vitro. J Surg Res. 1991 November; 51(5): 413-6.
Ghermay A P, Brady S, Havel R J, Harris H W, Rapp J H. Sepsis increases endocytosis of endotoxin into hepatocytes. Surgery. 1996 August; 120(2): 389-93; discussion 393-4.
Gilman's The Pharmacological Basis of Therapeutics. J. G. Hardman et al., 2001, McGraw-Hill Professional
Harris H W, Brady S E, Rapp J H. Hepatic endosomal trafficking of lipoprotein-bound endotoxin in rats. J Surg Res. 2002 July; 106(1): 188-95.
Harris H W, Eichbaum E B, Kane J P, Rapp J H. Detection of endotoxin in triglyceride-rich lipoproteins in vitro. J Lab Clin Med. 1991, 118:186-93.
Harris H W, Grunfeld C, Feingold K R, Rapp J H. Human very low density lipoproteins and chylomicrons can protect against endotoxin-induced death in mice. J Clin Invest. 1990 86:696-702.
Harris W S, Hustvedt B E, Hagen E, Green M H, Lu G, Drevon C A. N-3 fatty acids and chylonicron metabolism in the rat. J Lipid Res. 1997, 38:503-15.
Harris H W, Rockey D C, Chau P. Chylomicrons alter the hepatic distribution and cellular response to endotoxin in rats. Hepatology. 1998; 27:1341-8.
Harris W S. Chylomicron metabolism and omega 3 and omega 6 fatty acids. World Rev Nutr Diet. 1994; 76: 23-5.
Harris W S. omega 3 fatty acids and human chylomicron metabolism. World Rev Nutr Diet. 2001; 88: 163-7.
Harris H W, Kasravi F B. Lipoprotein-bound LPS induces cytokine tolerance in hepatocytes. J Endotoxin Res. 2003; 9(1): 45-50.
Harris W S, Muzio F., Fish oil reduces postprandial triglyceride concentrations without accelerating lipid-emulsion removal rates. Am J Clin Nutr. 1993, 58:68-74.
Harris H W, Grunfeld C, Feingold K R, Read T E, Kane J P, Jones A L, Eichbaum E B, Bland G F, Rapp J H. Chylomicrons alter the fate of endotoxin, decreasing tumor necrosis factor release and preventing death. J Clin Invest. 1993; 91:1028-34.
Kasravi F B, Brecht W J, Weisgraber K H, Harris H W. Induction of cytokine tolerance requires internalization of Chylomicron-Bound LPS into hepatocytes. J Surg Res. 2003; 115:303-9.
Kasravi F B, Welch W J, Peters-Lideu C A, Weisgraber K H, Harris H W. Induction of cytokine tolerance in rodent hepatocytes by chylomicron-bound LPS is low-density lipoprotein receptor dependent. Shock. 2003; 19:157-62.
Kumwenda Z L, Wong C B, Johnson J A, Gosnell J E, Welch W J, Harris H W. Chylomicron-bound endotoxin selectively inhibits NF-κK activation in rat hepatocytes. Shock. 2002; 18:182-8.
Kunz A N, Noel J M, Fairchok M P. Two cases of Lactobacillus bacteremia during probiotic treatment of short gut syndrome. J Pediatr Gastroenterol Nutr 2004; 38457.
Leaphart C L, Cavallo J, Gribar S C, Cetin S, Li J, Branca M F, Dubowski T D, Sodhi C P, Hackam D J. A critical role for TLR4 in the pathogenesis of necrotizing enterocolitis by modulating intestinal injury and repair. J. Immunol. 2007 Oct. 1; 179(7):4808-20
Lin H C, Hsu C H, Chen H L, Chung M Y, Hsu J F, Lien R, Tsao L Y, Chen C H, Su B H. Oral probiotics prevent necrotizing enterocolitis in very low birth weight preterm infants: a multicenter, randomized, controlled trial. Pediatrics 2008; 122:693.
J Lindsay, C van Montfrans, F Brennan, S van Deventer, P Drillenburg, H Hodgson, A to Velde and M Sol Rodriguez Pena, IL-10 gene therapy prevents TNBS-induced colitis, Gene Therapy (2002) 9:1715-1721
Locati M, Riboldi E, Bonecchi R, Transidico P, Bemasconi S, Haribabu B, Morris A J, Mantovani A, Sozzani S., Selective induction of phospholipase D1 in pathogen-activated human monocytes. Biochem J. 2001 358:119-25.
Lucas A, Cole T J. Breast milk and neonatal necrotizing enterocolitis. Lancet 1990; 336:1519.
Michele M. Kosiewicz, Cynthia C. Nast et al., Th1-type responses mediate spontaneous ileitis in a novel murine model of Crohn's disease. J Clin Investig 2001, 107:695-702
Masaya Kobayashi, Mi-Na Kweon, Hirotaka Kuwata, Robert D. Schreiber, Hiroshi Kiyono, Kiyoshi Takeda, and Shizuo Akira. Toll-like receptor-dependent production of IL-12p40 causes chronic enterocolitis in myeloid cell-specific Stat3-deficient mice J. Cline. Invest. 111:1297-1308 (2003).
Nadler E P, Dickinson E, Knisely A, Zhang X, Boyle P, Beer-Stolz D, Watkins S C, Ford H R. Expression of inducible nitric oxide synthase and interleukin-12 in experimental necrotizing enterocolitis. J Surg Res 2000; 92:71.
Nauli A M, Meng S, Yang Q, Li R, Jandacek R, Tso P. Intestinal alkaline phosphatase release is not associated with chylomicron formation. Am J Physiol Gastrointest Liver Physiol. 2003 284:G583-587.
Okayasu I, Hatakeyama S, Yamada M, Ohkusa T, Inagaki Y, Nakaya R. Related A novel method in the induction of reliable experimental acute and chronic ulcerative colitis in mice. Gastroenterology. 1990 98:694-702
Park Y, Harris W S. Omega-3 fatty acid supplementation accelerates chylomicron triglyceride clearance. J Lipid Res. 2003 44:455-63.
Park Y, Damron B D, Miles J M, Harris W S. Measurement of human chylomicron triglyceride clearance with a labeled commercial lipid emulsion. Lipids. 2001; 36:115-20.
Park Y, Grellner W J, Harris W S, Miles J M. A new method for the study of chylomicron kinetics in vivo. Am J Physiol Endocrinol Metab. 2000; 279:E1258-63.
Poelstra, K., Bakker, W. W., Klok, P. A., Hardonk, M. J., Meijer, D. K. F. (1997) A physiological function for AP: endotoxin detoxification. Lab Investig 76:319-327.
Poelstra, K., Bakker, W. W., Klok, P. A., Kamps, J. A. A. M., Hardonk, M. J., Meijer, D. K. F. (1997) Dephosphorylation of endotoxin by AP in vivo. Amer J Pathol 151, 1163-1169
Poelstra, K et al. (1994) Pharmaceutical composition comprising Phosphatase or a derivative thereof, WO1995-005456 A1
Poelstra, K et al., (1998): Diagnosis of sepsis using the LPS-binding moiety of AP as a diagnostic tool in patients with sepsis. WO2000-37943 A1
Read T E, Harris H W, Grunfeld C, Feingold K R, Kane J P, Rapp J H. The protective effect of serum lipoproteins against bacterial lipopolysaccharide. Eur Heart J. 1993; 14 Suppl K:125-9.
Read T E, Harris H W, Grunfeld C, Feingold K R, Calhoun M C, Kane J P, Rapp J H. Chylomicrons enhance endotoxin excretion in bile. Infect Immun. 1993; 61:3496-502.
Rennick, D. M. and Fort, M. M., Lessons From Genetically Engineered Animal Models XII. IL-10-deficient (IL-10−/−) mice and intestinal inflammation. Am J Physiol Gastrointest Liver Physiol 278: G829-G833, 2000.
Schanler R J, Shulman R J, Lau C. Feeding strategies for premature infants: beneficial outcomes of feeding fortified human milk versus preterm formula. Pediatrics 1999; 103:150.
Shao J S, Engle M, Xie Q, et al., Effect of tissue non-specific alkaline phosphatase in maintenance of structure of murine colon and stomach. Microsc Res Tech, 2000, 51:121-8
Strober, W, Nakamura K, Kitani, A: The SAMP1/Yit mouse: another step closer to modeling human inflammatory bowel disease. J Cline Investig 2001; 107:667-69
Whitehouse I S, Xu H, Shi Y, Noss L, Kaul S, Jones D W, Pritchard K A, Jr., Oldham K T, Gourlay D M. Mesenteric nitric oxide and superoxide production in experimental necrotizing enterocolitis. J Surg Res 2010; 161:1-8 (Epub 2009 Aug. 19).
Xu Q, Lu Z, Zhang X A novel role of alkaline phosphatase in protection from immunological liver injury in mice. Liver, 2002, 22:8-14
Xu H, Shi Y, Wang J, Jones D W, Weilrauch D, Ying R, Wakim B, Pritchard K A Jr. A heat shock protein 90 binding domain in endothelial nitric oxide synthase influences enzyme function. J Biol Chem 2007; 282:37567-74.

What is claimed is:

1. A medical food comprising enzymatically active alkaline phosphatase (AP) or an enzymatically active part thereof.

2. The medical food according to claim 1, wherein said medical food is an infant formula or (human) milk fortifier.

3. The medical food according to claim 1, designed to treat necrotizing enterocolitis.

4. The medical food according to claim 1, wherein the medical food comprises dry powder milk, a dry powder hydrolysate of milk, and/or dry powder soy protein.

5. The medical food according to claim 1, wherein the AP is a mammalian intestinal AP, a tissue non specific AP, a placental AP or a liver AP.

6. The medical food according to claim 1, wherein the AP is a recombinant or a modified recombinant AP.

7. The medical food according to claim 1 wherein the AP is of human or bovine origin.

8. The medical food according to claim 1 wherein the AP is enteric coated for oral administration and delivery to the gastro-intestinal tract mucosa.

9. The infant formula according to claim 2, comprising between 0.05 and 50,000 IU AP/g of dry powder and/or comprising between 0.8 and 800,000 IU AP/liter of liquid formula.

10. The fortifier according to claim 2, comprising between 0.2 and 200,000 IU AP/g of dry powder and/or comprising between 3 and 3,000,000 IU AP/liter of liquid fortifier.

11. A method for preventing and/or reducing necrotizing enterocolitis in a subject, comprising administering to the mucosal layer of the gastrointestinal tract of said subject an enzymatically active alkaline phosphatase.

12. The method according to claim 11 wherein the AP is administered orally.

13. The method according to claim 11, wherein the AP is administered via a suppository.

14. The method according to claim 11, wherein the AP is administered via a gastric tube, a transpyloric tube, and/or a rectal tube.

15. The method according to claim 11, wherein the AP is administered at least once per day.

16. The method according to claim 11, wherein the enzymatically active AP is present in a medical food according to claim 1.

17. A method for manufacturing an infant formula or milk fortifier for use in preventing and/or reducing inflammation of the gastro-intestinal tract mucosa, the method comprising the steps of adding a suitable amount of enzymatically active AP to a dry powder infant formula, a dry powder (human) milk fortifier, a liquid infant formula, a liquid (human) milk fortifier, and/or human milk.
